(19) United States
(12) Patent Application Publication
(10) Pub. No.: US 2019/0307811 A1
(43) Pub. Date: Oct. 10, 2019

(54) METHOD FOR TREATING AIRWAYS DISEASE

(71) Applicants: HUDSON INSTITUTE OF MEDICAL RESEARCH, Clayton, Victoria (AU); MONASH UNIVERSITY, Clayton, Victoria (AU)

(72) Inventors: Chrishan Surendran SAMUEL, Hawthorn East, Victoria (AU); Rebecca Seok Wai LIM, Clayton, Victoria (AU)

(73) Assignees: HUDSON INSTITUTE OF MEDICAL RESEARCH, Clayton, Victoria (AU); HUDSON INSTITUTE OF MEDICAL RESEARCH, Clayton, Victoria (AU); MONASH UNIVERSITY, Clayton, Victoria (AU); MONASH UNIVERSITY, Clayton, Victoria (AU)

(21) Appl. No.: 16/322,767

(22) PCT Filed: Aug. 4, 2017
(86) PCT No.: PCT/AU2017/050821

§ 371 Date: Feb. 1, 2019

(30) Foreign Application Priority Data
Aug. 4, 2016 (AU) 2016903060

(51) Int. Cl. A61K 35/50; A61K 38/22; A61P 11/06; A61P 11/00; A61K 35/28

(52) U.S. Cl. CPC A61K 35/50; A61K 38/2221; A61K 35/28; A61P 11/00; A61P 11/06

(57) ABSTRACT

The present disclosure teaches a method for treating airways disease including ameliorating symptoms of airway inflammation, airway remodeling and airway hyper-responsiveness. The method comprises the administration of an anti-fibrotic agent together with amnion epithelial cells or their functional equivalents or exosomes.

### CROSS-REFERENCE TO RELATED APPLICATIONS

This application is a 371 U.S. National phase of International Application No. PCT/AU2017/050821, filed Aug. 4, 2017, which is associated with and claims priority from Australian Provisional Patent Application No. 2016903060, filed on 4 Aug. 2016, entitled “A method of treatment”. The entire contents of both of the above applications are incorporated herein by reference.

### FIELD

The present disclosure teaches a method for treating airways disease including ameliorating symptoms of airway inflammation, airway remodeling and airway hyper-responsiveness.

### BACKGROUND

Bibliographic details of the publications referred to by author in this specification are collected alphabetically at the end of the description.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Lung disease, pulmonary syndromes and respiratory inflammatory disorders including bronchoconstriction and bronchospasm, collectively encompassed by the term “airways disease”, contribute to significant morbidity to afflicted subjects and are potentially life threatening. One particular condition of major significance is asthma. Asthma is an allergic airways disease (AAD) involving obstruction of the airways and bronchospasm (Levey et al. (2006) Prim. Care Respir. J. 15:20-34). The prevalence of asthma in developed countries has increased dramatically in recent decades (Myers and Tomasio (2011) Respir. Care 56:1389-1407), and has been estimated to affect 300 million people worldwide (Stanojevic et al. (2012) BMC Public Health 12:204). Asthma is responsible for one in every 250 deaths worldwide each year, as well as the loss of an estimated 15 million disability-adjusted life years (DALYs) and costs the health care system over $20 billion in Europe per year (Braman (2006) Chest 130:4S-12S).

Corticosteroids and β₂-adrenoreceptor agonists are the most widely-used treatments for asthma (Marceau et al. (2006) J. Allergy Clin. Immunol. 118:574-581). These therapies can suppress airway hyper-responsiveness (AHR; the constrictive response of the airways to a bronchoconstrictor [Holgate (2008) Clin. Exp. Allergy 38:872-897]) by targeting airway inflammation (AI) or AHR directly, respectively. However, they are limited in that they do not target the airway remodeling (AWR) associated with asthma. Airway remodeling involves structural changes within the lungs and airways including, but not limited to, the development of fibrosis that contributes to irreversible obstruction of the airways associated with asthma which can independently lead to airway hyper-responsiveness (Royce and Tang (2009) Curr. Mol. Pharmacol 2:169-181).

Stem cells possess attractive reparative properties (Ge et al. (2013) J. Cell. Biochem. 114:1595-1605) acting via paracrine signaling (Baraniak and McDevitt (2010) Regen. Med. 5:121-143), as well as increasing regulatory T cell (Treg) activity (Weiss (2014) Stem Cells 32:16-25). Stem cells have been considered for use in the treatment of airway remodeling. Stem cells have had some success in acute animal models of allergic airways disease to reduce inflammation and associated fibrosis, predominantly via immunomodulation (Royce et al. (2014) Pharm. therap. 141:250-260; Sun et al. (2012) Stem Cells 30:2692-2699; Moodley et al. (2010) Am. J. Rspir. Crit. Cre Med. 182:643-651). However, the use of stem cells have been less promising in chronic allergic airways disease models due to reductions in their proliferative, differentiation and reparative capabilities (Dolgachev et al. (2009) Am J. Path. 174:390-400). This is potentially attributed to the presence of extensive fibrosis in chronic AAD which impairs stem cell survival, migration to the target damaged tissue, proliferation and integration with resident tissue cells (Knight and Rossi (2010)Expert Rev. Respir. Med. 4:747-758).

Serelaxin (RLX) is the drug-based version of the major stored and circulating form of the human relaxin hormone, termed human gene-2 (H2) relaxin (relaxin-2). In addition to having rapid-occurring anti-fibrotic actions (Royce et al. (2014) supra) which are mediated through its cognate G protein coupled receptor, Relaxin Family Peptide Receptor-1 (RXFP1), serelaxin is currently being clinically assessed for its vasodilatory benefits in patients with acute heart failure (Teerlink et al. (2013) Lancet 281:29-39) and is also biologically active in mice (Samuel et al. (2007) Cell. Mol. Life Sci. 64:1539-1557). Serelaxin alone demonstrates therapeutic efficacy in various models of lung injury (Unemori et al. (1996) J. Clin. Invest. 98:2739-2745; Kenyon et al. (2003) Toxicol. Appl. Pharmacol. 186:90-100; Tozzi et al. (2005) Pulm. Pharm. Therap. 18:346-353; Huang et al. (2011) Am. J. Path. 179:2751-2765). In the setting of chronic AAD, systemic (Royce et al. (2009) Endocrinology 150:2692-2699) and intranasal (Royce et al. (2014) Clin. Exp. Allergy 44:1399-1408) treatment with serelaxin has been demonstrated to reduce excessive collagen deposition and airway epithelial thickening as well as prevent the development of AHR.

Combining serelaxin with human bone marrow-derived mesenchymal stem cells (MSCs) was found to improve the viability and proliferative capacity of these cells, and more effectively prevent obstructive nephropathy-induced renal fibrosis (Huuskes et al. (2015) FASEB J. 29:540-553). However, it was unknown whether a synergy could exist with other stem cell types for other conditions.

Human amnion epithelial stem cells (AECs) possess several potentially useful properties. They are non-immunogenic (Akle et al. (1981) Lancet 2:1003-1005) and can be easily and ethically harvested via non-invasive procedures from the amnion sac of the mature placenta (Miki et al. (2005) Stem Cells 23:1549-1559). AECs can reduce both inflammation and fibrosis in a chronic mouse model of bleomycin-induced interstitial lung injury (Moodley et al. (2010) supra), unlike MSCs which display only limited therapeutic efficacy when used in isolation in chronic disease settings (Huuskes et al. (2015) supra).

There is a need to develop more efficacious therapies for airways disease.

### SUMMARY

The present invention is predicated in part on the determination of a surprising synergy which exists between an anti-fibrotic agent active on lung tissue and amnion epithelial stem cells (AECs) or amniotic exosomes. An example of an anti-fibrotic agent is relaxin or a recombinant or functional derivative or variant form thereof. The present specification therefore teaches a therapeutic protocol to treat airways disease in a subject. The protocol comprises the co-administration, simultaneously or sequentially, in either order, of an anti-fibrotic agent and AECs or amniotic exosomes. The AECs are, in an embodiment, autologous to the subject being treated although can be allogeneic or in certain circumstances, xenogeneic, and the exosomes may be autologous, allogeneic or xenogeneic. The subject is generally a human subject in need of treatment. However, there are veterinary applications such as in the treatment of exercise induced pulmonary hemorrhage in racing animals such as race horses. Administration may be by any convenient route such as by intranasal and intrarespiratory routes but also by intravenous and the like.

In respect of human subjects, the airways disease includes acute and chronic allergic airways disease and reactive airways disease and the specific conditions of asthma, allergic rhinitis, chronic obstructive pulmonary disease (COPD), pulmonary fibrosis including idiopathic pulmonary fibrosis and other interstitial lung diseases, upper respiratory infection and reactive airways dysfunction syndrome. This is not intended to be an exhaustive list of airways diseases and all disease conditions having components of airway inflammation, airway remodeling and/or airway hyper-responsiveness, as well as conditions dependent thereon or associated therewith, are encompassed by the present invention. The present therapeutic protocol is further designed to treat the symptoms of these conditions, notably airway inflammation, airway remodeling and/or airway hyper-responsiveness. It is proposed herein that the combination of the ABCs or amniotic exosomes and an anti-fibrotic agent such as relaxin is useful in the prevention or reduction in risk of developing fibrotic lung tissue. The term “airways disease” is applicable to allergic and non-allergic inflammatory conditions of the lung and respiratory tract and either acute or chronic. Hence, diseases such as lung disease, reactive airways disease, pulmonary syndrome, pulmonary fibrosis including idiopathic pulmonary fibrosis and other interstitial lung diseases, respiratory inflammatory disorders including bronchoconstriction and bronchospasm are all encompassed by the term “airways disease”.

The AECs or exosomes may be administered separately to the anti-fibrotic agent or both may be co-formulated together or the AECs or exosomes may carry a form of the anti-fibrotic agent such as a recombinant form expressed in the AECs or a form introduced into exosomes. Conveniently, as indicated above, the anti-fibrotic agent is a relaxin such as human relaxin-2 or a recombinant form thereof such as serelaxin (also referred to herein as RLX) or a derivative of recombinant relaxin. The relaxin may be autologous, allogeneic or xenogeneic to the subject being treated and/or to the ABCs or exosomes.

A derivative of relaxin includes a single B chain functional derivative. An example of the latter is H2-(B7-33) as described by Hossain et al. (2016) Chem. Sci. 7:3805-3819. A derivative of relaxin also includes a functional truncated analog of both its A and B chains. An example of a truncated relaxin analog is H2-(A4-24)(B7-24) as described by Hossain et al. (2011) J. Biol. Chem. 286:37555-37565. Other derivatives of relaxin are also contemplated herein including N- and C-terminal truncates of the A and/or B chain as well as C-terminal amidated homologs, free acid forms and pyroglutamic acid analogs.

The subject protocol in relation to a relaxin component may alternatively or in addition employ a relaxin receptor activator or agonist. The receptor is RXFP1 and AECs express the RXFP1 receptor. Activators or agonists of the receptor include anti-RXFP1 antibodies, a pharmacophore or small molecule chemical or proteinaceous agonist and an estrogen-based compound such as estradiol.

As taught in the present Examples, the combination of AECs or amnion exosomes and relaxin is found to be more efficacious than a combination of mesenchymal stem cells (MSCs) and relaxin. ABCs or exosomes and relaxin normalize epithelial thickness and partially reverse fibrosis in a mouse model as well as ameliorate airway hyper-responsiveness. It is proposed herein that the anti-fibrotic agent such as relaxin provides an improved environment in which AEC-based or amnion-based therapies can be employed, enhancing the therapeutic and regenerative capacity of AECs expressing the relaxin-2 receptor, RXFP1 or amnion exosomes comprising same. It is further proposed that the therapeutic benefits of ABCs apply to amniotic exosomes. In this regard, the amniotic exosomes may be isolated from amniotic fluid or placental tissue or may be isolated from AEC lines including immortalized AEC lines. This includes the use of a bioreactor to generate amnion exosomes from AEC lines which are subsequently isolated and used in the present therapeutic protocol.

Pharmaceutical kits are also provided herein such as a kit comprising in compartmental form a first compartment comprising AECs or amniotic exosomes in a form which can be reconstituted in a pharmaceutically acceptable medium; a second compartment comprising an anti-fibrotic agent for use with lung tissue; wherein the AECs or exosomes are reconstituted in the pharmaceutically acceptable medium prior to use wherein the AECs and anti-fibrotic agent are administered to a subject simultaneously or sequentially in either order wherein the subject has airways disease.

Enabled herein is a formulation comprising AECs or amniotic exosomes and an anti-fibrotic agent and a pharmaceutical carrier, excipient and/or diluent. The anti-fibrotic agent includes a relaxin. The relaxin may be co-formulated with the ABCs or exosomes or contained or produced within the AECs or exosomes.

Further contemplated herein is the use of AECs or amniotic exosomes in combination with an anti-fibrotic agent in the manufacture of a medicament for the treatment of airways disease in a subject. In an embodiment, AECs or amniotic exosomes in combination with an anti-fibrotic agent are provided for use in the treatment of airways disease in a subject.

In an embodiment, the anti-fibrotic agent is a form of relaxin such as isolated naturally occurring relaxin, a recombinant relaxin such as serelaxin or a derivative single chain relaxin such as H2-(B7-33) [Hossain et al. (2016) supra] or truncated A and B chains of relaxin such as H2-(A4-24)(B7-24) [Hossain et al. (2011) supra]. In an embodiment, the relaxin receptor is alternatively, or in addition to relaxin, activated by an RXFP1 activator or agonist. The relaxin may be autologous, allogeneic or xenogeneic to the subject being treated, subject to safety testing to avoid rejection.

Abbreviations used herein are defined in Table 1.

TABLE 1
Abbreviations

| Abbreviation | Definition |
|---|---|
| AAD | Allergic airways disease (see also RAD) |
| AECs | Amnion epithelial stem cells |
| AHR | Airway hyper-responsiveness |
| AI | Airway inflammation |
| AWR | Airway remodeling |
| CO | Corn oil |
| COPD | Chronic obstructive pulmonary disease |
| EXO | Exosomes |
| H2-(B7-33) | Single B chain derivative of relaxin-2 (Hossain |
| | et al. (2016) supra) |
| H2(A4-24)(B7-24) | Derivative of relaxin with truncated A and B |
| | chains (Hossain et al. (2011) supra) |
| hRFs | Human renal fibroblasts |
| IP | Intraperitoneal |
| MSCs | Mesenchymal stem cells |
| NA | Naphthalene |
| OVA | Ovalbumin |
| RAD | Reactive airways disease (see also AAD) |
| RADs | Reactive airways dysfunction syndrome |
| RLX | Serelaxin (a recombinant form of human gene-2 |
| | relaxin) |
| RXFP1 | Relaxin family peptide receptor-1 (also known as |
| | LGR7) |
| TSLP | Thymic stromal lymphopoietin |

### BRIEF DESCRIPTION OF THE FIGURES

The drawings described herein are for illustrative purposes only of selected embodiments and not all possible implementations, and are not intended to limit the scope of the present disclosure.

Some figures contain color representations or entities. Color photographs are available from the Patentee upon request or from an appropriate Patent Office. A fee may be imposed if obtained from a Patent Office.

FIGS. 1a to c are a photographic representations of the expression of RXFP1 on AECs. AECs, and human renal fibroblasts (RFs; positive control) were stained for RXFP1 by immunofluorescence and nuclear counterstained with DAPI. Both AECs and hRFs had strong cytoplasmic staining for RXFP1. Staining was absent from negative control cells where primary antibody was substituted with an isotype control.

FIGS. 2a through e are images and graphical representations of the effects of RLX, MSCs, AECs and combination treatments on AI, epithelial thickness and subepithelial collagen. A) Representative images of Masson's trichrome-stained lung sections from each group studied demonstrating the extent of inflammatory cell infiltration within the bronchial wall. Scale bar=100 μm. Also shown is the median+interquartile range of peribronchial inflammation scores from 5 airways/mouse (where sections were scored based on the number and distribution of inflammatory cell aggregates on a scale of 0 (no visible inflammation) to 4 (severe inflammation)) (B; n=6/group); the mean+SEM eosinophil count per mL of BAL fluid (C; n=6 group); the mean+SEM epithelial thickness (μm²) relative to BM length (D; n=6 group); and the mean+SEM subepithelial collagen thickness (μm) relative to BM length (E; n=6/group). *P<0.05, **P<0.01, ***P<0.001 vs saline group; ^{#}P<0.05, ^{##}P<0.01, ^{###}P<0.001 vs OVA group; ^{¶}P<0.05, ^{¶¶}P<0.01 vs OVA+RLX group; ⁺P<0.05, ⁺⁺P<0.01 vs OVA+MSC group; ^{§}P<0.05 vs OVA+AEC group. OVA, ovalbumin.

FIGS. 3a and b are images and graphical representations of the effects of RLX, MSCs, AECs and combination treatments on epithelial damage. A) Representative images of thymic stromal lymphopoietin (TSLP)-stained lung sections from each group studied demonstrating the extent of epithelial damage. Scale bar=100 μm. Also shown is the mean+SEM number of TSLP-positive cells (indicated by arrows) within the epithelium/100 μm of BM length from 5 airways/mouse is also shown (B; n=6/group). **P<0.01, ***P<0.001 vs saline group; ^{#}P<0.05, ^{##}P<0.01 vs OVA group; ⁺P<0.05 vs OVA+MSC group.

FIGS. 4a and b is an image and a graphical representation of the effects of RLX, MSCs, AECs and combination treatments on goblet cell metaplasia. A) Representative images of Alcian blue periodic acid Schiff-stained lung sections from each treatment group demonstrating the number of goblet cells (indicated by arrows) present within the airway epithelium. Scale bar=100 μm. Also shown is the mean+SEM goblet cell count (represented as the number of goblet cells/100 μm BM length) from 5 airways/mouse (B; n=6/group). **P<0.01, ***P<0.001 vs saline group; ^{#}P<0.05 vs OVA group; ^{¶}P<0.05 vs OVA+RLX group; ⁺P<0.05 vs OVA+MSC group; ^{§}P<0.05 vs OVA+AEC group; ^{†}P<0.05 vs OVA+MSC+RLX group.

FIG. 5 is a graphical representation of the effects of RLX, MSCs, AECs and combination treatments on total lung collagen concentration. The total lung collagen content of each mouse was calculated via hydroxyproline analysis and then divided by the dry weight of the corresponding lung section analyzed to yield % collagen concentration. Shown is the mean+SEM % lung collagen concentration from each of the groups studied (n=6/group). ***P<0.001 vs saline group; ^{#}P<0.05, ^{##}P<0.01, ^{###}P<0.001 vs OVA group; ⁺⁺P<0.01 vs OVA+MSC group.

FIGS. 6a through d are images and graphical representations of the effects of RLX, MSCs, AECs and combination treatments on airway epithelial TGF-β1 expression and subepithelial myofibroblast accumulation. Representative images of immunohistochemistry-stained lung sections from each treatment group demonstrating the level of epithelial TGF-β1 expression and distribution within the airway epithelium (A) and subepithelial myofibroblast accumulation (C). Scale bar=100 μm (A, C). Also shown are the mean+SEM epithelial TGF-β1 staining (relative to that in the saline control group; which was expressed as 1) from 5 airways/mouse (B; n=5-6/group); and mean+SEM subepithelial myofibroblast number per 100 μm BM length (D; n=5-6/group) *p<0.05, **p<0.01, ***P<0.001 vs saline group; ^{#}P<0.05, ^{##}P<0.01, ^{###}P<0.001 vs OVA group; ^{¶}P<0.05 vs OVA+RLX group; ⁺P<0.05, ⁺⁺P<0.01 vs OVA+MSC group.

FIG. 7 is a graphical representation of the effects of RLX, MSCs, AECs and combination treatments on AHR. AHR was assessed by measuring airway resistance (via invasive plethysmography) in response to increasing doses of the bronchoconstrictor methacholine, delivered by nebulisation. Responses are expressed as the resistance change from the baseline response to saline. Shown is the mean±SEM of the airway resistance measured in response to each dose of methacholine, from each group studied (n=6/group). **P<0.01, ***P<0.001 vs saline group; ^{#}P<0.01, ^{###}P<0.001 vs OVA alone group; ^{¶}P<0.05 vs OVA+RLX group; ⁺⁺⁺P<0.001 vs OVA+MSC group; ^{§§}P<0.01, ^{§§§}P<0.001 vs OVA+AEC group. A legend for the graph lines is below:
[symbol] OVA, [symbol] OVA+MSCs, [symbol] OVA+AECs, [symbol] OVA+RLX, [symbol] OVA+AECs+RLX, [symbol] OVA+MSCs+RLX, [symbol] Saline.

FIG. 8 is a graphical representation showing mean±S.E.M peribronchial inflammation score from 5 airways/mouse (where sections were scored based on the number and distribution of inflammatory cell aggregates on a scale of 0 (no visible inflammation) to 4 (severe inflammation)); from n=6-8 animals per group. **P<0.01, ***P<0.001 vs saline/corn oil-treated uninjured control group; ^{##}P<0.01, ^{###}P<0.001 vs OVA/NA-treated chronic allergic airways disease (AAD) group; ^{¶}P<0.05, ^{¶¶}P<0.01 vs OVA/NA+5 μg AEC-derived exosome (EXO)-treated group; +p<0.05 vs OVA/NA+hAEC+RLX-treated group; ^{§§}P<0.01, ^{§§§}P<0.001 vs OVA/NA+RLX-treated group.

FIG. 9 is a graphical representation showing mean±S.E.M goblet cell count (represented as the number of goblet cells per 100 mm basement membrane (BM) length) from 5 airways/mouse; n=6-8 animals per group. **P<0.01, ***P<0.001 vs saline/corn oil-treated uninjured control group; ^{##}P<0.01, ^{###}P<0.001 vs OVA/NA-treated chronic allergic airways disease (AAD) group; ^{¶¶¶}P<0.001 vs OVA/NA+5 μg hAEC-derived exosome (EXO)-treated group; ^{‡‡‡}p<0.001 vs OVA/NA+25 μg hAEC-derived EXO-treated group; ⁺p<0.05 vs OVA/NA+5 μg hAEC-derived EXO+RLX-treated group; ⁺p<0.05 vs OVA/NA+hAEC+RLX-treated group; ^{§§}P<0.01, ^{§§§}P<0.001 vs OVA/NA+RLX-treated group.

FIG. 10 is a graphical representation showing mean±S.E.M TSLP-stained cell counts (per 100 μm basement membrane (BM) length) from 5 airways/mouse; n=8 animals per group. *p<0.05, **P<0.01, ***P<0.001 vs saline/corn oil-treated uninjured control group; ^{##}P<0.01, ^{###}P<0.001 vs OVA/NA-treated chronic allergic airways disease (AAD) group; ^{§§§}P<0.001 vs OVA/NA+RLX-treated group.

FIG. 11 is a graphical representation showing mean±S.E.M (A) epithelial thickness (μm²; relative to basement membrane (BM) length); and (B) subepithelial ECM thickness (μm; relative to BM length—a measure of fibrosis) from 5 airways/mouse; n=8 animals per group. *p<0.05, **P<0.01, ***P<0.001 vs saline/corn oil-treated uninjured control group; ^{##}P<0.01, ^{###}P<0.001 vs OVA/NA-treated chronic allergic airways disease (AAD) group; ^{¶}p<0.05; ^{¶¶}p<0.001, ^{¶¶¶}P<0.001 vs OVA/NA+5 μg hAEC-derived exosome (EXO)-treated group; ^{‡‡}p<0.01 vs OVA/NA+25 m μg hAEC-derived EXO-treated group; ^{§}P<0.05, ^{§§§}P<0.001 vs OVA/NA+RLX-treated group.

FIG. 12 is a graphical representation showing mean±S.E.M total lung collagen concentration (% lung collagen content/dry weight tissue—a measure of fibrosis); from n=7-8 animals per group. ^{#}P<0.05, ^{##}P<0.01, ^{###}P<0.00 1 vs OVA/NA-treated chronic allergic airways disease (AAD) group; ^{¶¶}p<0.01 vs OVA/NA+5 μg hAEC-derived exosome (EXO)-treated group; ^{‡}p<0.05 vs OVA/NA+25 μg hAEC-derived EXO-treated group; ^{§}P<0.05 vs OVA/NA+RLX-treated group.

FIG. 13 is a graphical representation showing mean±S.E.M TGF-β1-staining (expressing as % staining per area analyzed) from 5 airways/mouse; n=7-8 animals per group. *p<0.05, **P<0.01, ***P<0.001 vs saline/corn oil-treated uninjured control group; ^{##}P<0.01, ^{###}P<0.001 vs OVA/NA-treated chronic allergic airways disease (AAD) group; ^{¶¶¶}P<0.001 vs OVA/NA+5 μg hAEC-derived exosome (EXO)-treated group; ⁺p<0.05 vs OVA/NA+25 μg hAEC-derived EXO-treated group; ^{§§§}P<0.001 vs OVA/NA+RLX-treated group.

FIG. 14 is a graphical representation showing mean±S.E.M number of α-smooth muscle actin-stained myofibroblasts in the subepithelial region (per 100 μm basement membrane (BM) length) from 5airways/mouse; n=5-8 animals per group. *p<0.05, **P<0.01, ***P<0.001 vs saline/corn oil-treated uninjured control group; ^{##}P<0.01, ^{###}P<0.001 vs OVA/NA-treated chronic allergic airways disease (AAD) group; ^{¶}P<0.05 ^{¶¶¶}P<0.001 vs OVA/NA+5 μg hAEC-derived exosome (EXO)-treated group; ⁺p<0.05, ++p<0.01 vs OVA/NA+25 μg hAEC-derived EXO-treated group; ^{§§}P<0.01, ^{§§§}P<0.001 vs OVA/NA+RLX-treated group.

FIG. 15 is a graphical representation showing effects of the various groups evaluated on airway hyperresponsiveness (AHR). Airway resistance (reflecting changes in AHR) was assessed via invasive plethysmography in response to increasing doses of nebulized methacholine (a bronchoconstrictor). Results are expressed as resistance change from baseline. Shown is the mean±S.E.M. airway resistance to each dose of methacholine testes (n=5 animals per group). The effects of hAECs+recombinant human relaxin (RLX) or RLX alone are included for comparison. *p<0.05, **P<0.01, ***P<0.001 vs Saline/corn oil-treated uninjured control group; ^{##}P<0.01, ^{###}P<0.001 vs OVA/NA-treated chronic allergic airways disease (AAD) group; ^{¶}P<0.05 ^{¶¶}P<0.01, ^{¶¶¶}P<0.001 vs OVA/NA+5 μg hAEC-derived exosome (EXO)-treated group; ⁺p<0.05, ++p<0.01 vs OVA/NA+25 μg hAEC-derived EXO-treated group; ^{§}P<0.05, ^{§§}P<0.01 vs OVA/NA+RLX-treated group.
[symbol] OVA/NA(5), [symbol] OVA/NA+5 μg EXO(5), [symbol] OVA/NA+25 μg EXO(5); [symbol] OVA/NA+RLX(5), [symbol] OVA/NA+hAEC+RLX(5), [symbol] OVA/NA+5 μg EXO+RLX(5), [symbol] OVA/NA+25 μg EXO+RLX(5), [symbol] Saline/CO(5)

FIG. 16 is a graphical representation showing mean+S.E.M % interstitial fibrosis per field from 5 fields/mouse; from n=7 animals per group. ***P<0.001 vs saline (SAL)-treated uninjured control group; ^{###}P<0.001 vs bleomycin (BLM)-treated pulmonary fibrosis group.

FIG. 17 is a graphical representation showing mean+S.E.M subepithelial ECM thickness (μm; relative to BM length) from 5 airways/mouse; n=7 animals per group. *p<0.05, ***P<0.001 vs saline (SAL)-treated uninjured control group; ^{##}P<0.01, ^{###}P<0.001 vs bleomycin (BLM)-treated pulmonary fibrosis group; ^{¶}p<0.05 vs BLM+5 μg hAEC-derived exosome (EXO)-treated group; ⁺p<0.05 vs BLM +25 μg hAEC-derived EXO-treated group; ^{¤¤}P<0.01, ^{¤¤¤}P<0.001 vs BLM+Pirfenidone-treated group.

FIG. 18 is a graphical representation of mean±S.E.M % peribronchial inflammation score from 5 airways/mouse (where sections were scored based on the number and distribution of inflammatory cell aggregates on a scale of 0 (no visible inflammation) to 4 (severe inflammation)); from n=6-8 animals per group. *p<0.05, ***P<0.001 vs saline (SAL)-treated uninjured control group; ^{##}p<0.01, ^{###}P<0.001 vs bleomycin (BLM)-treated pulmonary fibrosis group; ⁺p<0.05, ⁺⁺p<0.01 vs BLM+hAEC+RLX-treated group.

Corresponding reference numerals indicate corresponding parts throughout the several views of the drawings.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

As used in the subject specification, the singular forms “a”, “an” and “the” include plural aspects unless the context clearly dictates otherwise. Thus, for example, reference to “a cell” includes a single cell, as well as two or more cells; reference to “an airways disease” includes reference to one or more specific disease forms of airways disease as well as one or more symptoms of airways disease; reference to “the disclosure” includes a single and multiple aspects taught by the disclosure; and so forth. Aspects taught and enabled herein are encompassed by the term “invention”. All such aspects are enabled within the width of the present invention. Any variants and derivatives contemplated herein are encompassed by “forms” of the invention.

Where a range of values is provided, it is understood that each intervening value, to the tenth of the unit of the lower limit unless the context clearly dictates otherwise, between the upper and lower limit of that range and any other stated or intervening value in that stated range is encompassed within the present invention. The upper and lower limits of these smaller ranges may independently be included in the smaller ranges which is also encompassed within the invention, subject to any specifically excluded limit in the stated range. Where the stated range includes one or both of the limits, ranges excluding either both of those included limits are also encompassed in the invention. The term “administration” in relation to two or more agents (e.g. cells, exosomes or a therapeutic entity) means the agents are given simultaneously or sequentially in either order. The term “simultaneously” includes but is not limited to when one agent (e.g. a cell or exosome) comprises another agent such as the therapeutic entity.

Generally, conventional methods of cell culture, stem cell biology, and recombinant DNA techniques within the skill of the art are employed in developing the present invention. Such techniques are explained fully in the literature, see, e.g. Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol NO. I, Cold Spring Harbor Laboratory (1998); and Harlow and Lane, Antibodies: A Laboratory Manual, Cold Spring Harbor Laboratory (1988).

The present invention is described primarily with reference to airways disease (AD) and therapy to ameliorate the potential for fibrosis to develop following injury or disease of the lung. The present invention is intended to cover use of an anti-fibrotic agent, in combination with AECs or amniotic exosomes to treat airways disease.

Abbreviations for various terms used in the Examples are defined in Table 1 and in the Detailed Description. The full terms or abbreviations are used in the Detailed Description.

The term “airways disease” means an acute or chronic respiratory lung disorder resulting from or exacerbated by or having symptoms of airway inflammation (AI), airway remodeling (AR) and/or airway hyper-responsiveness (AHR). The term “airways disease” and its abbreviation “AD” encompasses lung disease, reactive airways disease, pulmonary syndromes and respiratory inflammatory disorders including bronchoconstriction and bronchospasm conditions. These may be allergic or non-allergic in nature or cause. An example of airways disease is allergic airways disease (AAD) or reactive airways disease (RAD). Airways disease includes asthma, allergic rhinitis, chronic obstructive pulmonary disease (COPD), pulmonary fibrosis including idiopathic pulmonary fibrosis and other interstitial lung diseases, upper respiratory infection and reactive airways dysfunction syndrome (RADS). This is not intended to be an exhaustive list of conditions encompassed by the term “airways disease”. The present invention extends to respiratory and pulmonary conditions having one or more components of airway inflammation, airway remodeling and/or airway hyper-responsiveness. In an embodiment, the airways disease is an inflammatory airways disease which may or may not be allergic based. As indicated above, the airways disease may be acute or chronic.

The term “treatment” or “treating” means any therapeutic intervention in a subject, including: (i) prevention, in respect of causing the clinical symptoms not to develop; (ii) inhibition in respect of arresting the development of clinical symptoms; and/or (iii) relief in respect of causing the regression of clinical symptoms. In an embodiment, the aim is to ameliorate conditions or symptoms of airways disease such as airway inflammation, airway remodeling and/or airway hyper-responsiveness. Ultimately, the aim is to prevent, reduce the risk of or mitigate the development of fibrosis of lung or respiratory tract tissue. The respiratory tract includes airways.

The term “therapeutically effective amount” means a dosage sufficient to provide treatment including amelioration of symptoms of the airways disease condition. This will vary depending on the subject to be treated, the disease and/or symptomology of the disease, the method of delivery, and the desired clinical outcome. One major outcome is a reduction in inflammatory reduced airway remodeling and reduced incidence or risk of fibrosis or mitigation of the effects of fibrosis. In an embodiment, the effective amount of relaxin is 1 μg to 10,000 μg/kg/subject/dose. This includes per day, per 2-3 days, per week or per month. Any amount between and including 1 μg to 10,000 μg is encompassed by the present invention.

The present invention is predicated on the development of a therapeutic protocol for the treatment of airways disease and its various manifestations and symptoms including but not limited to inflammation, bronchoconstriction and/or bronchospasm, and ultimately fibrosis, airway remodeling and airway hyper-responsiveness. The therapeutic protocol comprises the administration to a subject in need of therapeutic intervention of:
(A) (i) AECs; o
(ii) amniotic exosomes; and
(B) an anti-fibrotic agent.
   The administration may be simultaneous or sequentially in either order. This includes when the anti-fibrotic agent is contained or produced within the AEC or exosome.

In an embodiment, the anti-fibrotic agent is relaxin or a recombinant or functional derivative form thereof. The anti-fibrotic actions of relaxin are summarized in Samuel et al. (2017) British Journal of Pharmacology 174:962-976. The AECs or exosomes and the anti-fibrotic agent may be provided in either order or co-administered at the same time or within seconds, minutes or hours or co-formulated together (e.g. the relaxin contained or produced within the AECs or exosomes). General methods for producing or containing biologics such as proteins in exosomes are provided in Sterzenbach et al. (2017) Molecular Therapy 25(6):1269-1278; Ha et al. (2016) Acta PharmaceuticaSinicaB 6(4):287-296; WO 2014/168548, and references therein.

The term “relaxin” means human relaxin, including intact full length relaxin or a portion of the relaxin molecule that retains biological activity [as described in U.S. Pat. No. 5,023,321; in an embodiment recombinant human relaxin (H2)] and other active agents with relaxin-like activity, such as Relaxin Like Factor (as described in U.S. Pat. No. 5,911,997 at SEQ ID NOS: 3 and 4, and column 5, line 27-column 6, line 4), relaxin and portions that retain biological activity analogs and portions that retain biological activity (as described in U.S. Pat. No. 5,811,395 at SEQ ID NOS: 1 and 2, and column 3, lines 16-40), and agents that competitively displace bound relaxin from a receptor, including those described in Agoulnik et al. (2017) British Journal of Pharmacology 174:977-989. Relaxin can be made by any method known to those skilled in the art, such as described in U.S. Pat. No. 4,835,251 and in U.S. Pat. N. 5,464,756. The term “relaxin-2” means H2 relaxin. Derivative forms of relaxin are described by Hossain et al. (2011) supra and Hossain et al. (2016) supra.

Further contemplated herein is the use of RXFP1 (formally LGR7) activating agents. The terms “RXFP1” and “LGR7” as used herein refer to the same entity which is a G protein-coupled receptor activated by relaxin H2 (also referred to as relaxin-2), as described in Ivell (2002) Science 295:637-638. Hence, taught herein is a therapeutic protocol comprising the administration simultaneously or sequentially in either order to a subject in need of therapeutic intervention of:
(A) (i) AECs; or
(ii) amniotic exosomes; and
(B) an anti-fibrotic agent and/or an activator or agonist of a receptor via which the anti-fibrotic agents acts on a cell.

In an embodiment, the anti-fibrotic agent is relaxin or a recombinant or derivative form thereof and the activator or agonist acts on RXFP1.

The term “RXFP1 activating agent” as used herein includes any molecules with the ability to activate RXFP1 in the same manner as relaxin, i.e., activation that provides an anti-fibrotic response similar to relaxin upon administration with AECs or amniotic exosomes as described in the methods herein. An RXFP1 activating agent includes, but is not limited to, a small molecule, a peptidomimetic, a pharmacophore, or an activating anti-RXFP1 antibody as well as an estrogen-based compound such as estradiol.

The medicament used in combination with AECs or exosomes therefrom includes a “pharmacophore”. Pharmacophores of the present invention mimic relaxin activity by interaction with an epitope of RXFP1 to which relaxin binds. Thus, a pharmacophore of the present invention has a shape (i.e. the geometric specifications) and electrochemical characteristics substantially as defined by the relaxin:RXFP1 complex. The term pharmacophore covers peptides, peptide analogs and small chemical molecules. Other derivatives of relaxin include relaxin analogs and relaxin mimetics (e.g. see WO 96/40185; WO 96/40186; Patil et al. (2017) British Journal of Pharmacology 174(10):950-961).

Without intending to limit the present invention to any theory or mode of action, the present invention is predicated in part on the finding that relaxin, through its G protein-coupled receptor (RXFP1) [described in Hsu et al. (2002) Science 295:671-674], enhances the reparative effects of AECs or amniotic exosomes. This enhances the overall anti-fibrotic effect and promotes a regenerative response in lung and respiratory tissue resulting from airways disease.

Hence, in an embodiment, taught herein is a therapeutic protocol to treat airways disease including its manifestations of airway inflammation, airway remodeling and/or airway hyper-responsiveness by the administration of:
(A) (i) a population of AECs; or
(ii) amniotic exosomes; and
(B) a relaxin or its recombinant form or a functional derivative thereof, optionally together with an RXFP1 activating agent or agonist.

In an embodiment, the airways disease is allergic airways disease or reactive airways disease. In an embodiment, the airways disease is asthma, allergic rhinitis, COPD, pulmonary fibrosis including idiopathic pulmonary fibrosis and other interstitial lung diseases, an upper respiratory infection or reactive airways dysfunction syndrome or symptoms thereof. In an embodiment, the treatment is to prevent or reduce the risk of developing or mitigating the effects of fibrosis of lung tissue, including respiratory tract tissue.

Accordingly, enabled herein is a method for treating airways disease in a subject, the method comprising administering to the subject a therapeutically effective amount of amnion epithelial stem cells (AECs) or amniotic exosomes together with an anti-fibrotic agent, the treatment being for a time and under conditions sufficient to ameliorate one or more of airway inflammation, airway remodeling and/or airway hyper-responsiveness.

Further taught herein is a method for treating allergic airways disease or reactive airways disease in a subject, the method comprising administering to the subject a therapeutically effective amount of amnion epithelial stem cells (AECs) or amniotic exosomes together with an anti-fibrotic agent, the treatment being for a time and under conditions sufficient to ameliorate one or more of airway inflammation, airway remodeling and/or airway hyper-responsiveness.

Still further taught herein is a method for treating airways disease selected from the list comprising or consisting of asthma, allergic rhinitis, COPD, pulmonary fibrosis including idiopathic pulmonary fibrosis and other interstitial lung diseases, upper respiratory infection and reactive airways dysfunction syndrome, in a subject, the method comprising administering to the subject a therapeutically effective amount of amnion epithelial cells (AECs) or amniotic exosomes together with an anti-fibrotic agent, the treatment being for a time and under conditions sufficient to ameliorate one or more of airway inflammation, airway remodeling and/or airway hyper-responsiveness.

Even yet further taught herein is a method for treating asthma in a subject, the method comprising administering to the subject a therapeutically effective amount of amnion epithelial stem cells (AECs) or amniotic exosomes together with an anti-fibrotic agent, the treatment being for a time and under conditions sufficient to ameliorate one or more of airway inflammation, airway remodeling and/or airway hyper-responsiveness.

Still yet another aspect enabled herein is a method for preventing or mitigating the effects of development of fibrosis of lung tissue including respiratory tissue in a subject, the method comprising administering to the subject a therapeutically effective amount of amnion epithelial cells (AECs) or amniotic exosomes together with an anti-fibrotic agent, the treatment being for a time and under conditions sufficient to ameliorate one or more of airway inflammation, airway remodeling and/or airway hyper-responsiveness. Reference to “mitigating” or “mitigation” includes reversal and amelioration of symptoms. In an embodiment, taught herein is a method for reversal or amelioration of symptoms the effects of development of fibrosis of lung tissue including respiratory tissue in a subject, the method comprising administering to the subject a therapeutically effective amount of amnion epithelial cells (AECs) or amniotic exosomes together with an anti-fibrotic agent, the treatment being for a time and under conditions sufficient to ameliorate one or more of airway inflammation, airway remodeling and/or airway hyper-responsiveness.

In an embodiment, the anti-fibrotic agent is an agent which can reduce fibrosis of lung tissue, which includes respiratory tract tissue. An example is relaxin (in particular relaxin-2 or H2 relaxin), a recombinant form thereof (such as serelaxin) or a functional derivative or variant of relaxin such as a single B chain derivative. An example of the latter is H2-(B7-33) [Hossain et al. (2016) supra]. Other derivatives include truncated A and B chain forms such as H2-(A4-24)(B7-24). Yet other derivatives include B chain N-terminal truncates, B chain C-terminal truncates, B chain N- and C-terminal truncates, C-terminal amidated homologs, free acid analogs and pyroglutamic acid analogs. These derivatives are described in Hossain et al. (2011) supra and Hossain et al. (2016) supra. Still other derivatives include analogs and variants such as described in WO 96/40185 and WO 96/40186. The relaxin or its various forms may also be administered with an RXFP1 activating or agonizing agent. Whilst the relaxin is generally autologous or allogeneic to the subject being treated, with suitable safety testing, xenogeneic relaxin may be used, i.e. a relaxin from one species used in another species. This also applies to the relationship between the AECs or exosomes and the relaxin.

Enabled herein is a method for treating airways disease in a subject, the method comprising administering to the subject a therapeutically effective amount of amnion epithelial cells (AECs) or amniotic exosomes together with a relaxin or a recombinant form thereof or a functional derivative thereof, the treatment being for a time and under conditions sufficient to ameliorate one or more of airway inflammation, airway remodeling and/or airway hyper-responsiveness.

Further taught herein is a method for treating allergic airways disease in a subject, the method comprising administering to the subject a therapeutically effective amount of amnion epithelial cells (AECs) or amniotic exosomes together with a relaxin or a recombinant form thereof or a functional derivative thereof, the treatment being for a time and under conditions sufficient to ameliorate one or more of airway inflammation, airway remodeling and/or airway hyper-responsiveness.

Still further taught herein is a method for treating airways disease selected from the list comprising or consisting of asthma, allergic rhinitis, COPD, pulmonary fibrosis including idiopathic pulmonary fibrosis and other interstitial lung diseases, upper respiratory infection and reactive airways dysfunction syndrome, in a subject, the method comprising administering to the subject a therapeutically effective amount of amnion epithelial cells (AECs) or amniotic exosomes together with a relaxin or a recombinant form thereof or a functional derivative thereof, the treatment being for a time and under conditions sufficient to ameliorate one or more of airway inflammation, airway remodeling and/or airway hyper-responsiveness.

Even yet further taught herein is a method for treating asthma in a subject, the method comprising administering to the subject a therapeutically effective amount of amnion epithelial cells (AECs) or amniotic exosomes together with a relaxin or a recombinant form thereof or a functional derivative thereof, the treatment being for a time and under conditions sufficient to ameliorate one or more of airway inflammation, airway remodeling and/or airway hyper-responsiveness.

Still yet another aspect enabled herein is a method for preventing or mitigating the effects of development of fibrosis of lung including respiratory tissue in a subject, the method comprising administering to the subject a therapeutically effective amount of amnion epithelial cells (AECs) or amniotic exosomes together with a relaxin or a recombinant form thereof or a functional derivative thereof, the treatment being for a time and under conditions sufficient to ameliorate one or more of airway inflammation, airway remodeling and/or airway hyper-responsiveness. This aspect includes pulmonary fibrosis such as idiopathic pulmonary fibrosis.

As indicated above, the list of airways diseases asthma, allergic rhinitis, COPD, pulmonary fibrosis including idiopathic pulmonary fibrosis and other interstitial lung diseases, upper respiratory infection and reactive airways dysfunctional syndrome is not intended to be exhaustive. As also indicated above, the relaxin or its various forms may also be used together with an RXFP1 activating or agonizing agent. Administration of a therapeutically effective amount of pharmaceutically active relaxin results in an enhancement of the resparative properties of AECs and amniotic exosomes resulting in a decrease in remodeling in response to airways disease and consequential lung damage.

In an embodiment, a method is provided for promoting or enhancing lung tissue healing and a prevention or reduction in fibrosis. Administration of an effective amount of a pharmaceutically active anti-fibrotic agent such as relaxin together with the AECs or exosomes to a subject in need thereof promotes lung tissue healing by at least from about 10% to 100% in a subject when compared to a suitable control, e.g. the amount of fibrotic tissue in the lung is decreased by at least from about 10% to 100% when compared to a suitable control. The expression, “from about 10% to 100%” means 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100. The efficacy of relaxin to promote lung tissue healing in combination with AECs or amniotic exosomes can be determined using any method known in the art including lung histopathology and various other assays including efficacy of breathing as well as biochemical markers.

The method of the present invention is suitable for treating a subject an individual who has been diagnosed with a disease which can lead to progressive lung fibrosis, who is suspected of having a disease related to progressive lung fibrosis, who is known to be susceptible and who is considered likely to develop a disease related to progressive lung fibrosis, or who is considered likely to develop a recurrence of a previously treated disease relating to progressive lung fibrosis. In addition, notwithstanding that human subjects are the principal beneficiaries of the subject therapeutic protocol, the protocol has application to racing animals such as horses, dogs and camels which can suffer from exercise induced pulmonary hemorrhage. Autologous, allogeneic or xenogeneic AECs, exosomes or relaxin can be employed, subject to suitable safety testing to minimize rejection. In an embodiment, autologous or allogenic forms of AECs exosomes and relaxin are used.

Existing evidence demonstrates the association of fibrosis with the lung disease process in a variety of airways diseases, including those asthma, allergic rhinitis, COPD, pulmonary fibrosis including idiopathic pulmonary fibrosis and other interstitial lung diseases, upper respiratory infection and reactive airways dysfunction syndrome. The subject therapeutic protocol employs the anti-fibrotic agent such as relaxin or its recombinant or functional derivative forms to enhance to reparative properties of AECs or amniotic exosomes. Hence, there is a synergy between the anti-fibrotic agent and the AECs or exosomes. The synergy extends to the additional or alternative use of an RXFP1 activating or agonizing agent. Such agents include anti-RXFP1 antibodies, pharmacophores, small chemical molecules and an estrogen-based compound such as estradiol.

The treatment of the airways disease can be determined by measuring one or more diagnostic parameters indicative of the course of the disease, compared to a suitable control. For comparison with animal models, a “suitable control” is an animal not treated with relaxin or other anti-fibrotic agent and either AECs or amniotic exosomes, or treated with the pharmaceutical formulation without one or other of these components or without either component. In the case of a human subject, a “suitable control” may be the individual before treatment, or may be a human (e.g. an age-matched or similar control) treated with a placebo.

Airways disease to be treated by the methods of the present invention may be due to a variety of diseases associated with lung fibroblast proliferation or the activation of extracellular matrix protein synthesis by lung fibroblasts. These diseases may be effectively treated in the present invention. Such diseases include asthmas, allergic rhinitis, COPD, upper respiratory infection and reactive airways dysfunction syndrome.

The anti-fibrotic agent and the AECs or amniotic exosomes may be maintained and administered separately, simultaneously or sequentially, or co-formulated prior to administration. Alternatively, the AECs or exosomes may comprise a form of the anti-fibrotic agent. For example, AECs may be genetically engineered to produce recombinant relaxin or a derivative form thereof. The exosomes may be manipulated to encapsulate relaxin, its recombinant form or a derivative of relaxin.

Formulations of the present invention are pharmaceutical formulations comprising a therapeutically effective amounts of pharmaceutically active relaxin or other anti-fibrotic agent and AECs or amniotic exosomes, and a pharmaceutically acceptable carrier excipient and/or diluent. The anti-fibrotic agent and the AECs or exosomes may be co-administered in the same formulation or administered simultaneously or sequentially in separate formulations or the AECs may be genetically engineered to express the anti-fibrotic agent such as relaxin. In addition, exosomes can be manipulated to comprise the anti-fibrotic agent. As indicated herein, an example of an anti-fibrotic agent is relaxin. For the sake of brevity, for the following description, the term “relaxin” includes its recombinant form or functional derivatives, unless otherwise specified.

Accordingly, enabled herein is a formulation comprising AECs or amniotic exosomes and an anti-fibrotic agent and one or more pharmaceutically acceptable diluents, excipients and/or carriers. In an embodiment, enabled herein is a formulation comprising AECs or amniotic exosomes and a relaxin and one or more pharmaceutically acceptable diluents, excipients and/or carriers. In an embodiment, taught herein is a formulation comprising amniotic exosomes and an anti-fibrotic agent such as relaxin and one or more pharmaceutically acceptable diluent, excipient and/or carrier. In an embodiment, the formulations are for use in the method of the present invention to treat airways disease, as herein before defined.

Relaxin, as an example of an anti-fibrotic proteinaceous molecule, may be administered as a polypeptide, or it may be expressed in AECs from a polynucleotide comprising a sequence which encodes relaxin. Relaxin suitable for use in the methods of the present invention can be isolated from natural sources, may be chemically or enzymatically synthesized, or produced using standard recombinant techniques known in the art. Examples of methods of making recombinant relaxin are found in various publications, including, e.g. U.S. Pat. Nos. 4,835,251; 5,326,694; 5,320,953; 5,464,756; and 5,759,807.

Relaxin suitable for use includes, but is not limited to, human relaxin, recombinant human relaxin, relaxin derived from non-human mammals, such as porcine relaxin, and any of a variety of variants of relaxin known in the art. Relaxin, pharmaceutically active relaxin variants, and pharmaceutical formulations comprising relaxin are well known in the art. See, e.g. U.S. Pat. Nos. 5,451,572; 5,811,395; 5,945,402; 6,780,836, 6,723,702, 5,166,191; and 5,759,807. In general, recombinant human relaxin (serelaxin) is identical in amino acid sequence to the naturally occurring product of the human H2 gene, consisting of an A chain of 24 amino acids and a B chain of 29 amino acids. Effective amounts of relaxin include from 1 to 10,000 μg/kg/subject. This includes from 1 μg/kg/subject to 1,000 μg/kg/subject. This includes from 10 μg/ to 800 μg/kg/subject or an amount inbetween any of these ranges. Dosage may be per day, week or month.

Relaxin-encoding nucleotide sequences are known in the art and can be used in AECs (e.g. GenBank Accession Nos. AF135824; AF076971; NM.sub.--006911; and NM.sub.-005059). The relaxin polynucleotides and polypeptides of the present invention can be introduced into an AEC by a gene delivery vehicle. The gene delivery vehicle may be of viral or non-viral origin (see generally, Jolly (1994) Cancer Gene Therapy 1:51-64; Kimura (1994) Human Gene Therapy 5:845-852; Connelly (1995) Human Gene Therapy 1:185-193; and Kaplitt (1994) Nature Genetics 6:148-153). Gene therapy vehicles for delivery of constructs including a coding sequence of a polynucleotide of the present invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest in AECs. Retrovirus vectors that can be employed include those described in EP 415 731; WO 90/07936; WO 94/03622; WO 93/25698; WO 93/25234; U.S. Pat. No. 5, 219,740; WO 93/11230; WO 93/10218; Vile and Hart (1993) Cancer Res. 53:3860-3864; Vile and Hart (1993) Cancer Res. 53:962-967; Ram et al. (1993) Cancer Res. 53:83-88; Takamiya et al. (1992) J. Neurosci. Res. 33:493-503; Baba et al. (1993) J. Neurosurg. 79:729-735; U.S. Pat. No. 4,777,127; and EP 345,242.

Gene delivery vehicles can also employ parvovirus such as adeno-associated virus (AAV) vectors. Representative examples include the AAV vectors disclosed by Srivastava in WO 93/09239, Samulski et al. (1989) J. Vir. 63:3822-3828; Mendelson et al. (1988) Virol. 166:154-165; and Flotte et al. (1993) Proc. Natl. Acad. Sci. USA 90:10613-10617.

Also of interest are adenoviral vectors, e.g. those described by Berkner (1988) Biotechniques 6:616-627; Rosenfeld et al. (1991) Science 252:431-434; WO 93/19191; Kolls et al. (1994) Proc. Natl. Acad. Sci. USA 91:215-219; Kass-Eisler et al. (1993) Proc. Natl. Acad. Sci. USA 90:11498-11502; WO 94/12649, WO 93/03769; WO 93/19191; WO 94/28938; WO 95/11984 and WO 95/00655.

Other gene delivery vehicles and methods may be employed, including polycationic condensed DNA linked or unlinked to killed adenovirus alone, for example Curiel (1992) Hum. Gene Ther. 3:147-154; ligand linked DNA, for example see Wu (1989) J. Biol. Chem. 264:16985-16987; eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip (1994) Mol. Cell Biol. 14:2411-2418, and in Woffendin (1994) Proc. Natl. Acad. Sci. 91:1581-1585.

Further non-viral delivery suitable for use includes mechanical delivery systems such as the approach described in Woffendin et al. (1994) supra. This type of approach can be used to introduce relaxin protein to exosomes. Moreover, the coding sequence and the product of expression of such can be delivered through deposition of photopolymerized hydrogel materials. Other conventional methods for gene delivery that can be used for delivery of the coding sequence to AECs include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/11033.

The relaxin may also be incorporated within the exosomes or AECs. See, for example, WO2014/168548.

Administration of a pharmaceutical composition comprising AECs or amniotic exosomes together with or separately to the anti-fibrotic agent (e.g. relaxin [or the AECs or exosomes comprising the anti-fibrotic agent]), may be performed by any convenient means known to one skilled in the art. Routes of administration include, but are not limited to, respiratorally, intranasally, intratracheally, nasopharyngeally, intravenously, intraperitoneally, intrathoracically, subcutaneously, intradermally, intramuscularly, intraoccularly, intrathecally, rectally and by a slow or sustained release implant.

The pharmaceutical forms suitable for injectable use include sterile aqueous solutions or dispersions.

Sterile injectable solutions in the form of dispersions are generally prepared by incorporating the various sterilized active ingredients into a sterile vehicle which contains the amniotic exosomes.

For parenteral administration, the active components may be formulated with a pharmaceutical carrier and administered as a suspension. Illustrative of suitable carriers are water, saline, dextrose solutions, fructose solutions, ethanol, or oils of animal, vegetative or synthetic origin. The carrier may also contain other ingredients, for example, preservatives, buffers and the like. When the active components are being administered intrathecally, they may also be formulated in cerebrospinal fluid.

The active components of the subject invention can also be administered in sustained delivery or sustained release mechanisms, which can deliver the exosomes internally over a period of time. For example, biodegradeable microspheres or capsules or other biodegradeable polymer configurations capable of sustained delivery of the amniotic exosomes can be included in the formulations of the invention (e.g. Putney and Burke (1998) Nat Biotech 16:153-157).

In preparing pharmaceutical compositions of the present invention, a variety of formulation techniques can be used and manipulated to alter biodistribution. A number of methods for altering biodistribution are known to one of ordinary skill in the art. Examples of such methods include protection of the exosomes in vesicles composed of substances such as proteins, lipids (for example, liposomes), carbohydrates, or synthetic polymers. For a general discussion of pharmacokinetics, see, e.g., Remington: The Science and Practice of Pharmacy 21^{st} ed. (2006).

In general, a dose of relaxin may be from about 0.1 to 500 μg/kg of body weight per day or week simultaneously or sequentially with the AECs or exosomes. Such an amount includes 0.1, 0.5, 1, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 μg/kg per day or week or such other interval as determined by the physician. In an embodiment, it is desirable to obtain a serum concentration of relaxin at or above about 1.0 ng/ml, from about 0.5 to about 50 ng/ml, from about 1 to about 20 ng/ml. For administration to a 70 kg person, a dosage may be in a range of from about 2 μg to about 2 mg per day, from about 10 μg to 500 μg per day, or from about 50 μg to about 100 μg per day. The amount of relaxin administered will, of course, be dependent on the subject and the severity of the affliction, the manner and schedule of administration, the number of AECs or exosomes also delivered and the judgment of the prescribing physician.

RXFP1 and relaxin form a complex with a particular molecular interaction, and pharmacophores fitting this geometric and chemical description can be used in the present methods to activate RXFP1 at the RXFP1-relaxin interface. These activators can be used in place of or in addition to relaxin in the presently described methods of the invention. Identifying pharmacophores of the invention requires the identification of small molecules, peptides, and the like that mimics the positive image of the residues that comprise the relaxin binding site on RXFP1. A successful compound binds to RXFP1, activating and thereby the effects similar to those seen upon relaxin administration.

Candidate molecules as RXFP1 activating pharmacophores can encompass numerous chemical classes, including, but not limited to, peptides and small molecules. Candidate pharmacophores can comprise functional groups necessary for structural interaction with proteins, particularly hydrogen bonding, and typically include at least an amine, carbonyl, hydroxyl or carboxyl group, generally at least two of the functional chemical groups. The candidate pharmacophores often comprise cyclical carbon or heterocyclic structures and/or aromatic or polyaromatic structures substituted with one or more of the above functional groups. Candidate inhibitor pharmacophores are also found among biomolecules including, but not limited to: polynucleotides, peptides, saccharides, fatty acids, steroids, purines, pyrimidines, derivatives, structural analogs or combinations thereof.

Candidate RXFP1 activating pharmacophores can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacologically relevant scaffolds may be subjected to directed or random chemical modifications, such as acylation, alkylation, esterification, amidification, etc. to produce structural analogs.

Identification of structural aspects of proteins involved in relaxin-RXFP1 complex formation can define a tertiary structure to be used in an assay to design pharmacophores that modulate molecules and/or protein:protein interactions in the complex. Specifically, a dataset of compounds (small molecules, peptides, etc) having a particular tertiary structure can be identified using techniques known in the art, such as medicinal chemistry, combinatorial chemistry and molecular modeling, to determine molecules that are likely to bind to the atoms or groups of atoms of a protein involved in the binding of RXFP1 and relaxin. Optionally, factors such as hydrophobicity and hydrophilicity, placement of the functional residues in a structural motif, and the like may also be taken into account.

In an embodiment of the assay of the invention, the assay involves: (1) matching compounds in a library with the binding site regarding spatial orientation; (2) screening candidate compounds visually using computer generated molecular display software; and (3) experimentally screening actual compounds against RXFP1 in the presence and absence of relaxin to determine compounds which enhance signalling activity through LGR7.

Once the functional residues of the target protein are identified, this portion of the molecule can serves as a template for comparison with known molecules, e.g. in a database such as Available Chemicals Database (ACD, Molecular Design Labs, 1997), or it may be used to design molecules de novo. In one example, the initial group of identified molecules may contain tens or hundreds of thousands or more of different non-peptide organic compounds. A different or supplemental group may contain millions of different peptides which could be produced synthetically in chemical reactions or via bacteria or phage. Large peptide libraries and methods of making such are disclosed in U.S. Pat. No. 5,266,684, issued Nov. 30, 1993, and U.S. Pat. No. 5,420,246, issued May 30, 1995, which are incorporated herein by reference. Libraries of non-peptide organic molecules are disclosed in PCT publication WO 96/40202.

The initial library of molecules is screened via computer generated modeling, e.g. computer models of the compounds are matched against a computer model of the relaxin ligand binding site on RXFP1 to find molecules which mimic the spatial orientation and basic structure of the relaxin epitope. This screening should substantially reduce the number of candidate molecules relative to the initial group.

The screened group is then subjected to further screening visually using a suitable computer program which makes viewable images of the molecules. The resulting candidate molecules are then actually tested for their ability to enhance relaxin-RXFP1 complex formation and resulting activation of RXFP1.

Further taught herein is a pharmaceutical kit a pharmaceutical kit comprising in compartmental form a first compartment comprising AECs or amniotic exosomes in a form which can be reconstituted in a pharmaceutically acceptable medium; a second compartment comprising an anti-fibrotic agent for use with lung tissue; wherein the AECs or exosomes are reconstituted in the pharmaceutically acceptable medium prior to use wherein the AECs and anti-fibrotic agent are administered to a subject simultaneously or sequentially in either order wherein the subject has airways disease.

In an embodiment, the anti-fibrotic agent is relaxin (relaxin-2) or its recombinant form such as serelaxin or functional derivative forms (e.g. a single B chain derivative such as H2-(B7-33) or an A and B chain truncated such as H2-(A4-24)(B7-24)). Other derivatives of relaxin are contemplated herein as described in Hossain et al. (2011) supra and Hossain et al. (2016) supra. Alternatively, the AECs or exosomes comprise the relaxin.

The present invention also contemplates the use of AECs or amniotic exosomes in combination with an anti-fibrotic agent in the manufacture of a medicament for the treatment of airways disease in a subject. As indicated above, an example of an anti-fibrotic agent is relaxin, as hereinbefore described.

Also taught here are AECs or amniotic exosomes and an anti-fibrotic agent for use in the treatment of airways disease in a subject. As above, an example of an anti-fibrotic agent is relaxin or a recombinant or derivative form thereof. The action of the relaxin or its forms may also be enhanced by the use of RXFP1 activating or agonizing agents as discussed above.

Notwithstanding that AECs are referred to in the alternative to amniotic exosomes, the present invention does not exclude AECs and exosomes being co-formulated or co-mixed for use in accordance with the subject therapeutic protocol.

The amniotic exosomes may conveniently be obtained from a bioreactor culturing AECs. The AECs may be isolated at different gestational stages resulting in the amniotic exosomes having different reparative properties or spectra. The AECs or exosomes may be autologous, allogeneic or, in some circumstances if rejection can be minimized, xenogeneic to the subject being treated.

Notwithstanding the benefits of using AECs or amniotic exosomes and an anti-fibrotic agent such as relaxin to treat airways disease, the present disclose extends to the use of MSCs and an anti-fibrotic agent such as relaxin to treat these conditions. Accordingly, the present disclosure extends to a method of treating airways disease in a subject, the method comprising administering to the subject, a therapeutically effective amount of mesenchymal stem cells (MSCs) together with an anti-fibrotic agent, the treatment being for a time and under conditions to ameliorate one or more of airway inflammation, airway remodeling and/or airway hyperresponsiveness. In an embodiment, the anti-fibrotic agent is a relaxin.

The airways diseases for treatment using MSCs are as disclosed herein. Also taught herein is a formulation comprising MSCs and an anti-fibrotic agent such as relaxin when used for treating airways disease.

### EXAMPLES

Aspects disclosed herein are further described by the following non-limiting Examples.

### Materials and Methods

### Animals

Six-to-eight week-old female Balb/c mice were obtained from Monash Animal Services (Monash University, Clayton, Victoria, Australia) and housed under a controlled environment, on a 12 hour light/12 hour dark lighting cycle with free access to water and lab chow (Barastock Stockfeeds, Pakenham, Victoria, Australia). All mice were provided an acclimatization period of 4-5 days before any experimentation.

### Induction of Chronic AAD

To assess the individual vs combined effects of MSCs, AECs and RLX (serelaxin) in chronic AAD, a chronic model of ovalbumin (OVA)-induced AAD was established in mice (n=36). Mice were sensitized with two i.p. injections of 10 μg of Grade V chicken egg OVA (Sigma-Aldrich, Mo., USA) and 400 μg of aluminium potassium sulphate adjuvant (alum; AJAX Chemicals, NSW, Australia) in 500 μl of 0.9% w/v normal saline solution (Baxter Health Care, NSW, Australia) on day 0 and day 14. They were then challenged by whole body inhalation exposure (nebulization) to aerosolized OVA (2.5% w/v in 0.9% w/v normal saline) for thirty minutes, three times a week, between days 21 and 63, using an ultrasonic nebulizer (Omron NE-U07; Omron, Kyoto, Japan). Control mice (n=6) were given i.p injections of 500 μl 0.9% w/v saline and nebulized with 0.9% w/v saline instead of OVA.

### Intranasal Treatment of Stem Cells and/or RLX

On day 64 (1 day following completion of induction of the chronic AAD model), mice were lightly anaesthetized with isoflurane (Baxter Health Care, NSW, Australia) and held in a semisupine position while intranasal administration of the appropriate treatment took place. The following treatments were administered to mice from days 64-77, over a two-week period: OVA Alone: mice (n=6) received 50 μL of phosphate buffered saline (PBS; the vehicle for MSCs and AECs) (25 μL per nare), using an automatic pipette on days 64 and 71 over the two week treatment period; and used as the injury control group.

RLX alone: mice (n=6) daily received 50 μL (25 μL per nare) of a 0.8 mg/mL (equivalent to 0.5 mg/kg/day) RLX solution (Corthera Inc, San Carlos, Calif., USA; a subsidiary of Novartis Pharma AG, Basel, Switzerland), via intranasal delivery (Royce et al. (2014) supra; Royce et al. (2015) Stem Cell Res. 15:495-505), over the two-week treatment period (from days 64-77).

MSCs alone: MSCs (from the Tulane Center for Stem Cell Research and Regenerative Medicine; New Orleans, La., USA; pooled from n=3-4 healthy donors) were characterized and cultured as previously described (Wise et al. (2014) am. J. Phyisol. Renal Physiol. 306:F1222-F1235); and used between passages 3-6 for the outlined studies. Prior to administration, 1×10⁶ MSCs were resuspended in 50 μL of PBS and intranasally administered to mice (n=6) once weekly; 25 μL per nare on days 64 and 71 of the two-week treatment period (Royce et al. (2015) supra).

AECs alone: AECs (from term placentas; pooled from 2-3 separate donors; and characterized previously [Murphy et al. (2010) Curr. Prot. Stem Cell Biol. chapter 1, Unit 1E6]) were obtained and reconstituted overnight (once thawed from being stored in liquid nitrogen) before use in Dulbecco's modified Eagle's medium (DMEM)/F-12 media containing 10% v/v FBS. 1×10⁶ AECs were resuspended in 50 μL of PBS and administered to a fourth sub-group of mice (n=6), on days 64 and 71, as detailed above for MSC treatment of mice.

Combination treatment: Two further sub-groups of mice (n=6/group) received either intranasal administration of MSCs and RLX or AECs and RLX (as described above for each treatment) over the two-week treatment period. RLX was administered approximately 15-20 minutes prior to MSC or AEC administration in each case.

### Invasive Plethysmography

On day 78 (24 hours after the final intranasal administration of PBS or various treatments detailed), mice were anesthetized with an i.p injection of ketamine 10 mg/kg and xylazine 2 mg/kg BW (in 0.9% w/v saline). Tracheostomy was then performed and anesthetized mice were then positioned in the chamber of the Buxco Fine Pointe plethysmograph (Buxco, Research Systems, Wilmington, N.C., USA). The airway resistance of each mouse was then measured (reflecting changes in AHR) in response to increasing doses of nebulized acetyl-β-methylcholine chloride (methacholine; Sigma Aldrich, Mo., USA), delivered intratracheally, from 3.125-50 mg/ml over 5 doses, to elicit bronchoconstriction. The change in airway resistance calculated by the maximal resistance after each dose minus baseline resistance (PBS alone) was plotted against each dose of methacholine evaluated.

### Bronchoalveolar Lavage (BAL) and Tissue Collection

Following invasive plethysmography, BAL fluid was collected by pooling 3×0.5 mL lavages with ice cold PBS and stored in 300 μL of 5% v/v FBS, at −80° C. Lung tissues were then isolated and rinsed in cold PBS before divided into four separate lobes. The largest lobe was fixed in 10% v/v neutral buffered formaldehyde overnight and processed to be cut and embedded in paraffin wax. The remaining three lobes were snap-frozen in liquid nitrogen for various other assays.

### Lung Histopathology

Once the largest lobe from each mouse had been processed and paraffin-embedded, each tissue block was serially-sectioned (3 μm thickness) and placed on charged Mikro Glass slides (Grale Scientific, Ringwood, Victoria, Australia) and subjected to various histological stains or immunohistochemistry. To assess inflammation score, one slide from each mouse (total of 42) underwent Mayer's haematoxylin and eosin (Amber Scientific, Midvale, Western Australia, Australia) (H&E) staining. Similarly, to assess epithelial thickness and sub-epithelial collagen deposition, another set of slides underwent Masson's trichrome staining. To assess goblet cell metaplasia, a third set of slides underwent Alcian blue periodic acid Schiff (ABPAS) staining. The H&E, Masson trichrome and ABPAS-stained sections were morphometrically analyzed, as detailed below.

### Immunohistochemistry (IHC) and Immunofluorescence (IF)

IHC was used to detect TGF-β1 (using a polyclonal antibody; sc-146; Santa Cruz Biotechnology, Santa Cruz, Calif., USA; 1:1000 dilution), α-smooth muscle actin (α-SMA; a marker of myofibroblast differentiation; using a monoclonal antibody; M0851; DAKO Antibodies, Glostrup, Denmark; 1:200 dilution) and thymic stromal lymphopoietin (TSLP; a marker for epithelial damage; using a polyclonal antibody; ABT330; EMD Millipore Corp'. Temecula, Calif., USA; 1:1000 dilution). Primary antibody staining was detected using the DAKO EnVision anti-rabbit or anti-mouse kits and 3,3′-Diaminobenzidine (DAB) chromogen, while negative controls, which were exposed to the EnVision kits in the absence of any primary antibody, were also included. All slides were then counter-stained with haematoxylin.

If was performed on AECs and human renal fibroblasts (RFs; used as a positive control; provided by Kolling Institute of Medical Research, University of Sydney, NSW, Australia) cultured on chamber slides to detect RXFP1 (using a polyclonal antibody to RXFP1; HPA027067; Sigma-Aldrich, Castle Hill, NSW, Australia; 1:200 dilution). Primary antibody was detected using a goat anti-rabbit Alexa Fluor (Registered Trade Mark) 555 secondary antibody (Invitrogen, Carlsbad, Calif., USA). Nuclei was visualized with 4′6-diamindion-2-phenylindole (DAPI), while an isotype (negative) control was also included.

All IHC-stained slides were scanned by Monash Histology Services, Clayton, Victoria, Australia using ScanScope AT Turbo (Aperio, Calif., USA) for morphometric analysis; while IF-stained slides were imaged using a HyD confocal microscope (Leica SP8 Confical Invert, Monash Micro Imaging, Clayton Victoria, Australia).

### Morphometric Analysis

Masson's trichrome-, ABPAS- and IHC-stained slides underwent morphometric analysis as follows. Five airways (of 150-300 μm in diameter) per slide were randomly selected and analyzed using Aperio ImageScope software (Aperio, Calif., USA). Masson's trichrome-stained slides underwent semi-quantitative peri-bronchiolar inflammation scoring, where the experimenter was blinded and scored individual airways from 0 (no detectable inflammation surrounding the airway) to 4 (widespread and massive inflammatory cell aggregates, pooled size ˜0.6 mm²), as previously described (Royce et al. (2015) supra). Masson's trichrome-stained slides also underwent analysis for epithelial thickness and subepithelial collagen by measuring the thickness of the epithelium and the subepithelial collagen layer (stained blue); which were expressed as μm²/μm of basement membrane (BM) length.

ABPAS-, α-SMA- and TSLP-stained slides were analyzed for goblet cell metaplasia, myofibroblast number and damaged epithelial cells, respectively, by counting the number of positively stained goblet cells, α-SMA-positive cells and TSLP-positive cells per 100 μm of BM length. TGF-β1-stained slides were analyzed for TGF-β1 protein expression by running an algorithm to assess strong positively-stained pixels within the airway epithelium. Results were expressed as the number of strong positive pixels per total area (mm²) of epithelium; and then relative to that of the saline-treated control group, which was expressed as 1.

### Hydroxyproline Assay

The second largest lung lobe from each mouse was processed as previously described (Royce et al. (2009) supra; Royce et al. (2014) supra; Royce et al. (2015) supra) for the measurement of hydroxyproline content, which was determined from a standard curve of purified trans-4-hydroxy-L-proline (Sigma-Aldrich). Hydroxyproline values were multiplied by a factor of 6.94 ((based on hydroxyproline representing ˜14.4% of the amino acid composition of collagen in most mammalian tissues (Gallop and Paz (1975) Physiol. Revs. 55:418-487); to extrapolate total collagen content, which in turn was divided by the dry weight of each corresponding tissue to yield percent collagen concentration.

### Statistical Analysis

All statistical analysis was performed using GraphPad Prism v6.0 (GraphPad Software Inc., Calif., USA) and expressed as the mean±SEM. AHR results were analyzed by a two-way ANOVA with Bonferroni post-hoc test. The remaining data were analyzed via a one-way ANOVA with Neuman-Keuls post-hoc test for multiple comparisons between groups. In each case, data were considered significant with a p-value less than 0.05.

### Induction of Chronic Allergic Airways Disease (AAD) Incorporating Epithelial Damage

The following is particularly relevant to Example 9. An ovalbumin (OVA)-induced model of chronic AAD incorporating epithelial damage was established in mice (n=56). Mice, as described above, were sensitized with two intraperitoneal (IP) injections of 10 μg of Grade V chicken egg OVA (Sigma-Aldrich, Mo., USA) and 400 μg of aluminium potassium sulphate adjuvant (alum; AJAX Chemicals, NSW, Australia) on day 0 and 14. They were then challenged by whole body inhalation exposure (nebulization) to aerosolized OVA (2.5% w/v in 0.9% w/v normal saline) for thirty minutes, three times a week, between days 21 and 63, using an ultrasonic nebulizer (Omron NE-U07; Omron, Kyoto, Japan). The mice then received a single IP injection of the Clara cell-specific cytotoxin, naphthalene (NA; 200 mg/kg body weight; Sigma-Aldrich, St Louis, Mo., USA) on day-64 (1-day after the last OVA nebulization period; OVA+NA group) and left for a further three days (to induce and exacerbate airway epithelial damage). For control mice (n=8), however, instead of OVA, they were given IP injections of 500 μL 0.9% w/v saline and nebulized with 0.9% w/v saline; and instead of NA, they were injected with corn oil (CO; the vehicle for NA).

In terms of intranasal treatment of hAECs, exosomes and/or RLX, on day 68 (1 day following completion of induction of the chronic OVA/NA-induced AAD model), mice were lightly anaesthetized with isoflurane (Baxter Health Care, NSW, Australia) and held in a semi-supine position while intranasal administration of the appropriate treatment took place. The following treatments were administered daily or once-weekly to mice, from days 68-74:

OVA/NA alone (injury control group): a-sub-group of mice (n=8) received 50 μL of phosphate buffered saline (PBS; the vehicle for AECs and AEC-derived exosomes; 25 μL per nare) daily, using an automatic pipette from days 68-74.

RLX: a separate sub-group of mice (n=8) daily received 50 μL (25 μL per nare) of a 0.8 mg/mL (equivalent to 0.5 mg/kg/day) RLX solution (Corthera Inc, San Carlos, Calif., USA; a subsidiary of Novartis Pharma AG, Basel, Switzerland) from days 68-74.

AECs+RLX: As intranasal administration of AECs (1×10⁶/mouse)+RLX (0.8 mg/ml) had previously been shown to reduce the OVA-induced airway inflammation and abrogate the OVA-induced airway fibrosis and airway hyperresponsiveness (Royce et al. (2016) Clinical Science 130:3151-65), the combination therapy was applied to a third sub-group of OVA/NA-injured mice and used as a positive control group. Frozen AECs (from term placentas; pooled from 2-3 separate donors) were thawed in a 37° C. water bath, then resuspended in PBS. 1×10⁶ AECs were resuspended in 50 μL of PBS and administered to a separate sub-group of mice (n=8), on day 68. RLX (0.8 mg/ml) was administered daily (50 μl/mouse) from days 68-74, as detailed above.

AEC-derived exosomes: A fourth and fifth sub-group of OVA/NA-injured mice (n=8 per sub-group) were each administered with 50 μL of PBS containing either 5 μg or 25 μg of AEC-derived exosomes, respectively. Exosomes were intranasally-administered on day 68 only.

AEC-derived exosomes+RLX: A sixth and seventh sub-group of OVA/NA-injured mice (n=8 per sub-group) were each administered with 50 μL of PBS containing either 5 μg or 25 μg of AEC-derived exosomes, respectively; on day 68 only. These mice also received daily intranasal administration of RLX (0.8 mg/ml), from days 68-74, as detailed above.

Invasive plethysmography as described above was performed on 75 mice.

Tissue collection, lung histopathology, immunohistochemistry (IHC), morphometric analysis, hydroxyproline assay and statistical analysis were as described above.

### Induction of Bleomycin-Induced Pulmonary Fibrosis

The following is relevant to Example 10. A bleomycin (BLM)-induced model of pulmonary fibrosis was established in mice (n=49). Mice were intranasally (IN) administered with 20 μg of bleomycin sulphate (Hospira, Melbourne, Victoria, Australia) in 50 μl of saline (equivalent to 400 μg/ml; 25 μl per nare) on days 0 and 7. For control mice (n=7), however, instead of BLM, they were given IN administration (50 μL per mouse) of 0.9% w/v saline on days 0 and 7. All mice were then left untreated for a further two weeks (until day 21).

On day 22 (1 day following completion of induction of the BLM-induced pulmonary fibrosis model), mice were lightly anaesthetized with isoflurane (Baxter Health Care, NSW, Australia) and held in a semi-supine position while IN administration of the appropriate treatment took place. The following treatments were administered daily or once-weekly to mice, from days 22-28.

BLM alone (injury control group): a-sub-group of mice (n=7) received 50 μL of phosphate buffered saline (PBS; the vehicle for Pirfenidone, AECs and AEC-derived exosomes; 25 μL per nare) daily, using an automatic pipette from days 22-28,

Pirfenidone: a separate sub-group of mice (n=7) received 1 mg of Pirfenidone (Tocris Bioscience, Noble Park, Victoria, Australia) per day (equivalent to 50 mg/kg/day) by twice daily oral gavage, from days 22-28.

AECs+recombinant human relaxin (RLX): As per the chronic AAD model, a third group of BLM-injured mice received intranasal administration of AECs (1×10⁶/mouse)+RLX (0.8 mg/ml). Frozen AECs (from term placentas; pooled from 2-3 separate donors) were thawed in a 37° C. water bath, then resuspended in PBS. 1×10⁶ AECs were resuspended in 50 μL of PBS and administered to a separate sub-group of mice (n=7), on day 22. RLX (0.8 mg/ml) was IN-administered daily (50 μl/mouse) from days 22-28.

AEC-derived exosomes: A fourth and fifth sub-group of BLM-injured mice (n=7 per sub-group) were each administered with 50 μL of PBS containing either 5 μg or 25 μg of AEC-derived exosomes, respectively. Exosomes were IN-administered on day 22 only.

AEC-derived exosomes+RLX: A sixth and seventh sub-group of BLM-injured mice (n=7 per sub-group) were each administered with 50 μL of PBS containing either 5 μg or 25 μg of AEC-derived exosomes, respectively; on day 22 only. These mice also received daily IN administration of RLX (0.8 mg/ml), from days 22-28, as detailed above.

Invasive plethysmography, tissue collection, lung histopathology, morphometric analysis and statistics are as described above.

### Example 1

### Expression of RXFP1 on AECs

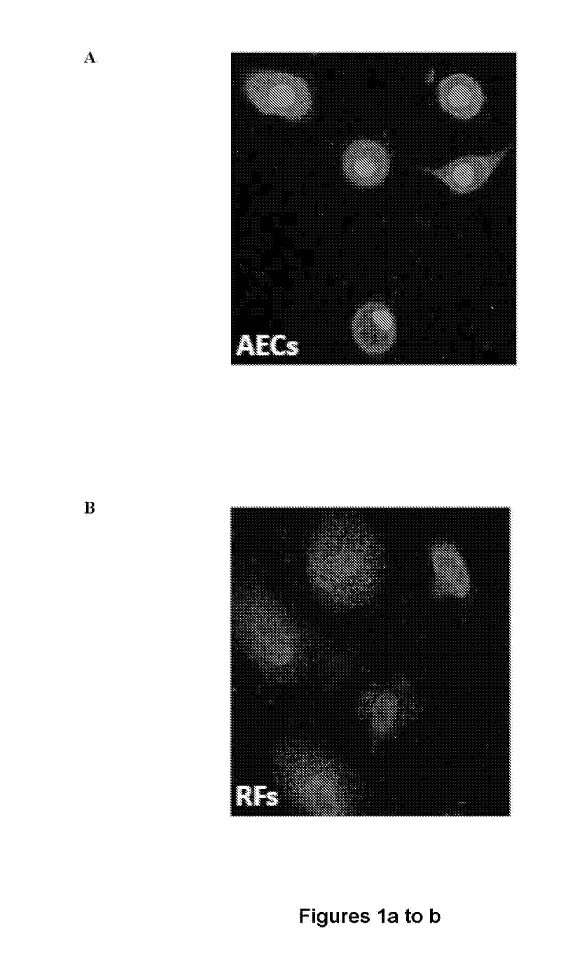

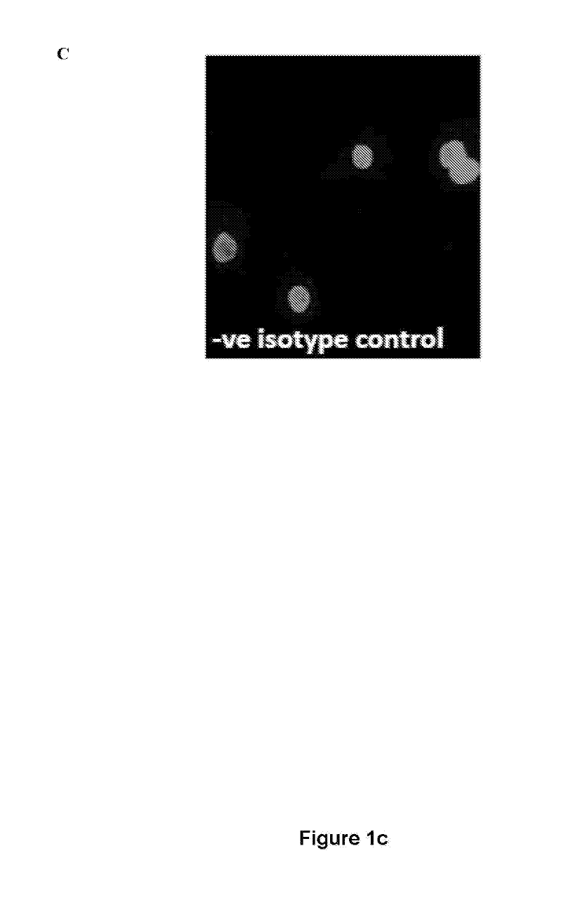

AECs and human renal fibroblasts (RFs; positive control) stained positively for RXFP1 by immunofluorescence and nuclear counterstaining with DAPI (FIG. 1). Both AECs and RFs had strong cytoplasmic staining for RXFP1. Staining was absent from negative control cells where primary antibody was substituted with an isotype control.

### Example 2

### Effects of RLX, MSCs, AECs and Combination Treatments on Airway Inflammation (AI)

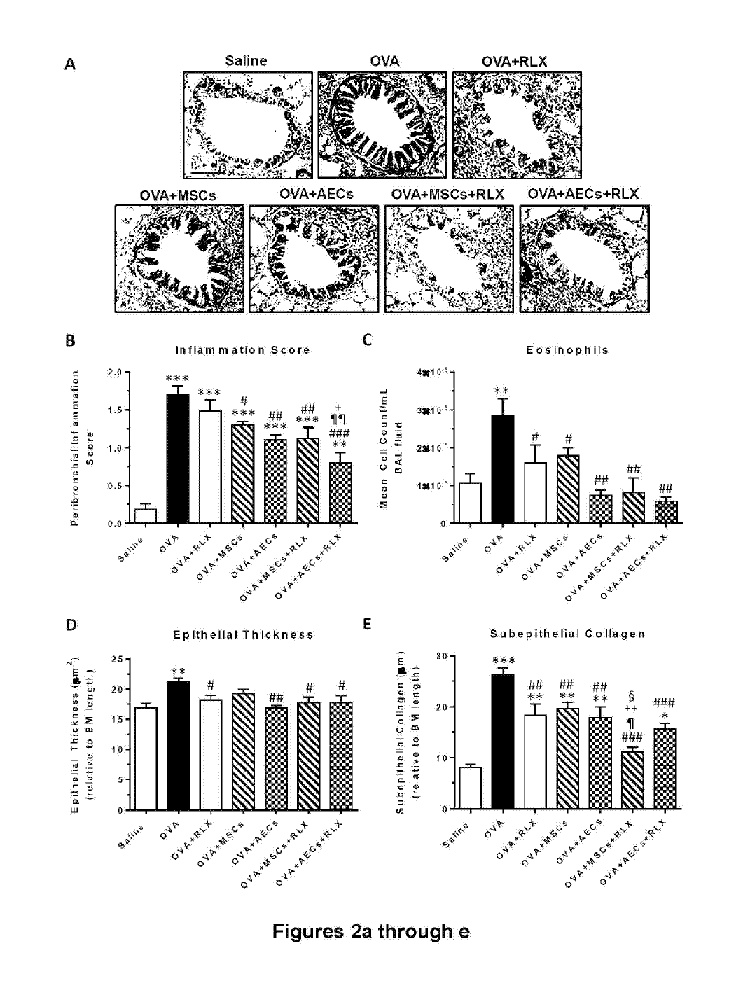

Peri-bronchial inflammation score from Masson's trichrome-stained images (FIGS. 2A and 2B) and eosinophil infiltration from the BAL fluid (FIG. 2C) were used as measures of AI. The peri-bronchial inflammation score of OVA-treated mice (1.70±0.60) was significantly increased compared to that of saline controls (0.15±0.40; P<0.001 vs saline group; FIG. 2B). This confirmed that the chronic model of AAD had been established in OVA-treated mice. RLX-treatment of animals did not significantly affect the OVA-induced increase in inflammation score (1.55±0.83; P<0.001 vs saline group), while MSCs alone (1.30±0.20; P<0.05 vs OVA group; P<0.001 vs saline group) only modestly but significantly reduced peribronchial inflammation (FIG. 2B). In comparison, AECs alone (1.05±0.28), MSCs+RLX (1.00±0.40) and AECs+RLX (0.90±0.70) were able to significantly reduce OVA-induced inflammation score by ˜40-50%; with the greatest effect observed with AECs+RLX (P<0.001 vs OVA group; P<0.01 vs OVA+RLX group; P<0.05 vs OVA+MSC group). However, neither AECs alone nor the combination treatments were able to reduce peri-bronchial inflammation to that seen in saline-treated controls (all P<0.01 vs saline group; FIG. 2B).

OVA-treated mice also had significantly increased numbers of eosinophils (2.85+0.45/mL of BAL fluid) compared to that from saline-treated controls (1.06×10₅±0.25×10₅/mL of BAL fluid; P<0.01 vs saline group; FIG. 2C). Both RLX (1.60×10₅±0.47×10₅/mL of BAL fluid) and MSCs alone (1.79×10₅±0.21×10₅/mL of BAL fluid) partially, but significantly, decreased eosinophil infiltration to a similar degree (both P<0.05 vs OVA alone), but to a level which did not differ to that measured in saline-treated controls (FIG. 2C). In comparison, AECs alone (7.37×10₄±1.46×10₄/mL of BAL fluid), and the combined effects of MSCs+RLX (8.19×10₄±3.79×10₄/mL of BAL fluid) or AECs+RLX (5.92×10₄±1.05×10₄/mL of BAL fluid) completely abrogated eosinophil infiltration (all P<0.01 vs OVA group; no different to saline group), again to a level which was no longer different to that measured in saline-treated controls (FIG. 2C).

### Example 3

### Effects of RLX, MSCs, AECs and Combination Treatments on Epithelial Thickness and Damage

The epithelial thickness of OVA-treated mice (21.20±0.60 μm²) was significantly increased compared to that of saline-treated controls (16.89±0.76 μm²; P<0.01 vs saline group; FIG. 2D). Treatment with MSCs alone (19.23±0.73 μm²) only induced a trend towards reducing the OVA-induced increase in epithelial thickness, although this value was not statistically different to that measured from saline-treated control mice. In comparison, RLX alone (18.19±0.74 μm²; p<0.05 vs OVA alone), AECs alone (16.90±0.39 μm²), MSCs+RLX (17.75±0.87 μm²) and AECs+RLX (18.21±2.19 μm²) all normalized the OVA-induced increase in epithelial thickness back to that seen in saline-treated control mice (all P<0.05 vs OVA alone group; no different to saline group; FIG. 2D).

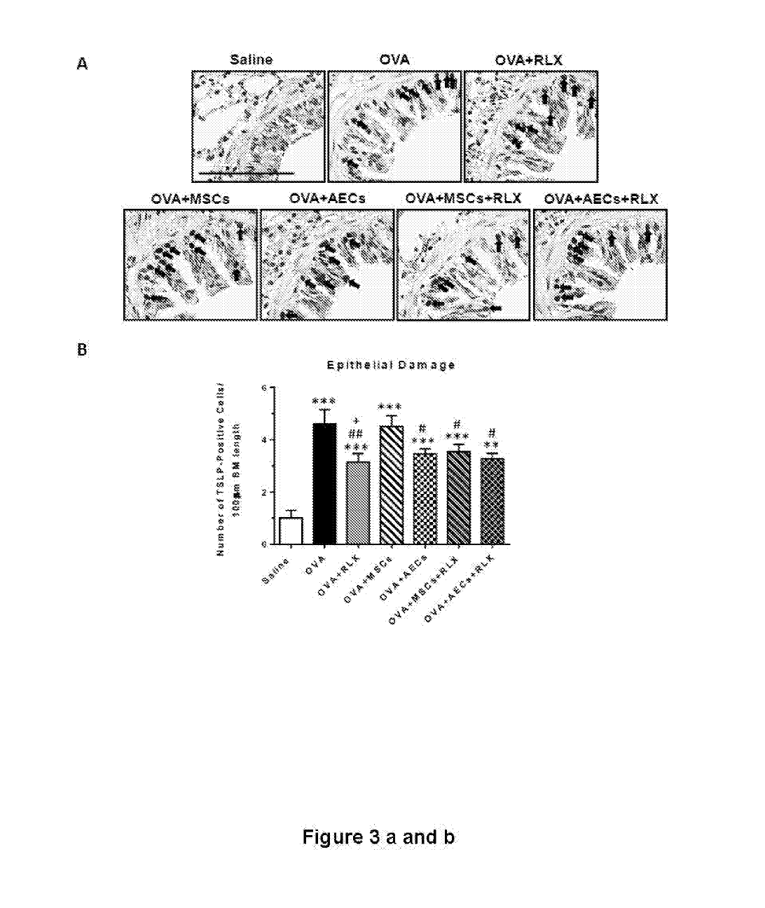

TSLP was used a marker of epithelial damage and the number of TSLP-positive cells within the airway epithelium was significantly higher in OVA-treated mice (4.61±0.53) compared to that in their saline-treated counterparts (1.00±0.30; P<0.001 vs saline group). MSCs alone (4.50±0.42) were unable to reduce the OVA-induced increase in TSLP expression. However, RLX alone (3.14±0.34), AECs alone (3.50±0.19) and the combined effects of MSCs+RLX (3.53±0.28) or AECs+RLX (3.30±0.27) all significantly reduced TSLP expression levels compared to that in the OVA alone group (all P<0.05 vs OVA alone group); but not to levels measured in saline-treated control mice (all P<0.01 vs saline group) [Figure 3]. Of note, RLX alone also reduced TSLP expression levels compared to the effects of MSCs alone (P<0.05 vs OVA+MSC group; FIG. 3).

### Example 4

### Effects of RLX, MSCs, AECs and Combination Treatments on Goblet Cell Metaplasia

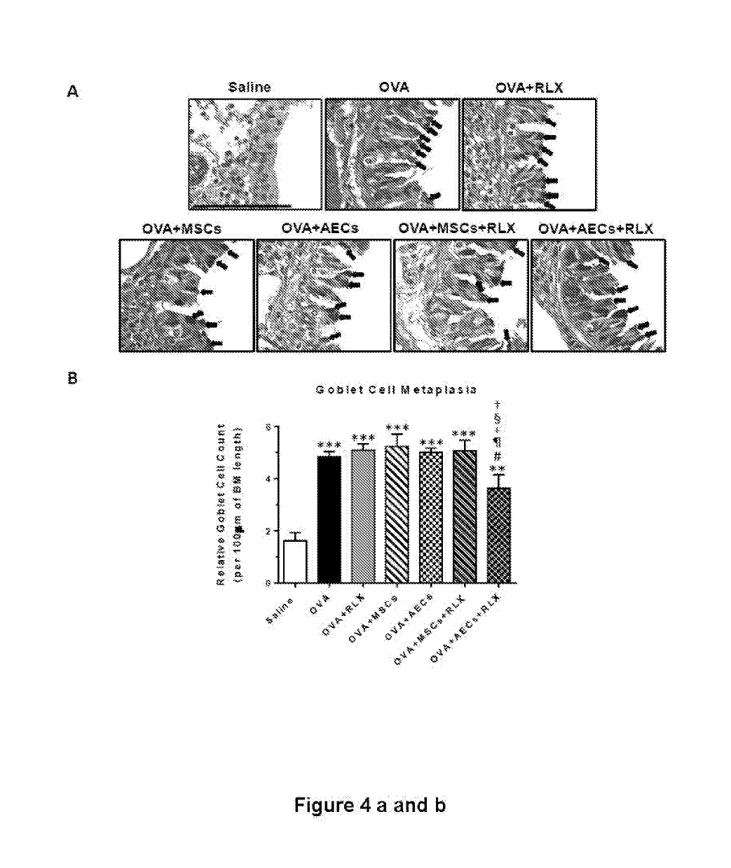

Goblet cell metaplasia was analyzed from ABPAS-stained lung sections and was significantly increased in OVA-treated mice (4.83±0.20) compared to that measured from saline-treated counterparts (1.62±0.32; P<0.001 vs saline group; FIG. 4). Neither RLX alone (5.08±0.25), MSCs alone (5.23±0.47), AECs alone (5.00±0.16) or MSCs+RLX (5.06±0.41) affected the OVA-induced increase in goblet cell numbers (all P<0.001 vs saline group). In comparison, only the combined effects of AECs+RLX (4.39±0.28) significantly reduced the OVA-induced increase in goblet cell numbers (P<0.05 vs OVA alone; OVA+RLX; OVA+MSCs, OVA+AECs; and OVA+MSCs+RLX groups; FIG. 4); but not fully back to levels measured from saline-treated controls (P<0.01 vs saline group).

### Example 5

### Effects of RLX, MSCs, AECs and Combination Treatments on Airway Fibrosis

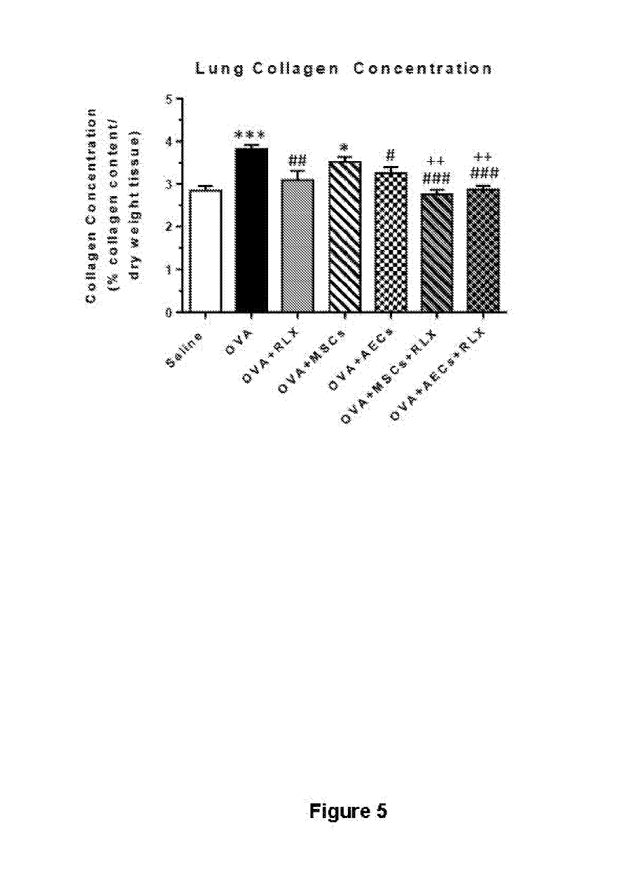

Airway fibrosis was measured by analyzing subepithelial collagen deposition from Masson's trichrome-stained lung sections (FIG. 2E) and hydroxyproline analysis of total lung collagen concentration (FIG. 5). Subepithelial collagen (relative to basement membrane length) was significantly increased in OVA-treated mice (26.26±1.37 μm) compared to that measured from saline-treated controls (8.07±0.58 μm; P<0.001 vs saline group; FIG. 2E). RLX alone (18.27±2.26 μm), MSCs alone (19.67±1.17 μm) or AECs alone (17.87±2.150 μm) significantly reduced the OVA-induced aberrant increase in subepithelial collagen deposition to a similar extent (by ˜35-45%; all P<0.01 vs OVA alone group; FIG. 2E). Mice treated with AECs+RLX had a further reduction in subepithelial collagen deposition (13.72±1.25 μm) compared to that measured in OVA-injured mice (by ˜70%; P<0.001 vs OVA group; P<0.05 vs saline group), while the OVA-induced increased in subepithelial collagen deposition was normalized (11.12±0.93 μm) by the combined effects of MSCs+RLX (P<0.001 vs OVA group; no different to saline group); and to a greater extent than either therapy alone (P<0.05 vs RLX alone or AECs alone; P<0.01 vs MSCs alone; FIG. 2E).

Similar findings were observed with measurements of total lung collagen concentration, which was significantly increased in OVA-treated mice (3.82±0.10%) compared to corresponding measurements obtained from saline-treated controls (2.84±0.11%; P<0.001 vs saline group); and significantly reduced by RLX alone (3.08±0.23%; P<0.01 vs OVA alone group) or AECs alone (3.26±0.14%; P<0.05 vs OVA alone group), but not MSCs alone (3.52±0.12%) (FIG. 5). Strikingly, the combined effects of MSCs+RLX (2.75±0.11%; P<0.001 vs OVA alone group) or AECs+RLX (2.90±0.04%; P<0.01 vs OVA alone group) completely reversed the OVA-induced increase in lung collagen concentration, back to levels measured in saline-treated control animals (FIG. 5). The combined effects of MSCs+RLX or AECs+RLX also reduced total lung collagen concentration to a greater extent that MSCs alone (both P<0.01 vs OVA+MSC group; FIG. 5).

### Example 6

### Effects of RLX, MSCs, AECs and Combination Treatments on Airway Epithelial TGF-β1 Expression

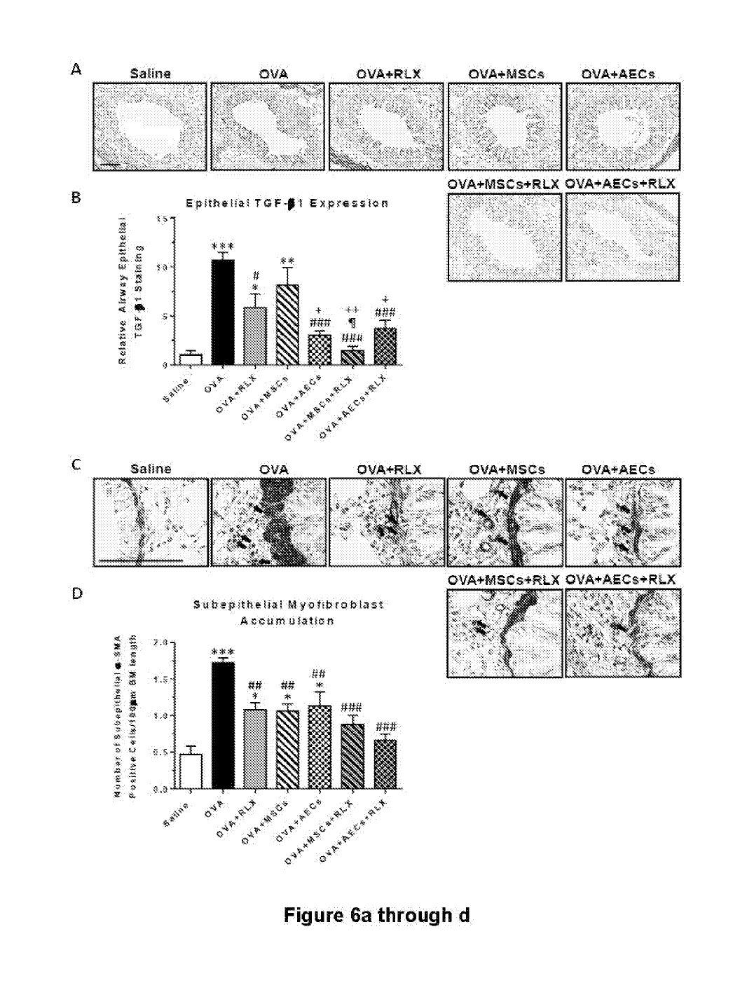

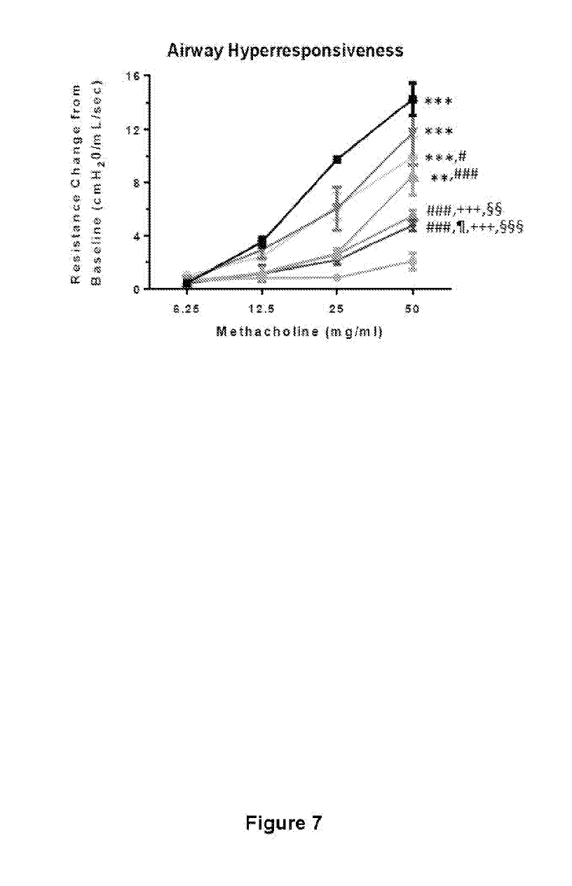

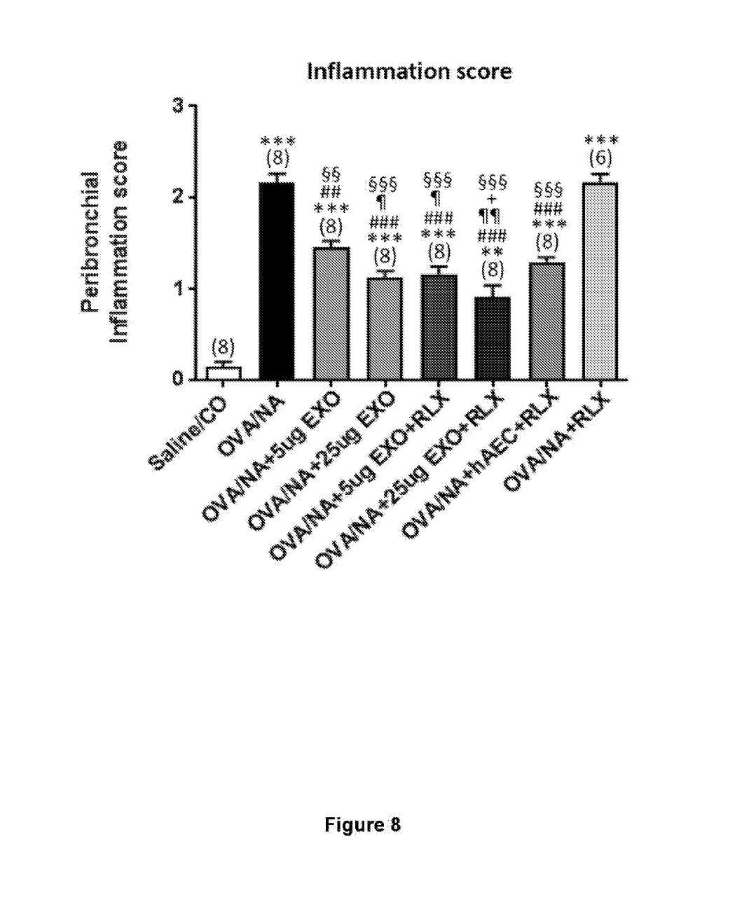

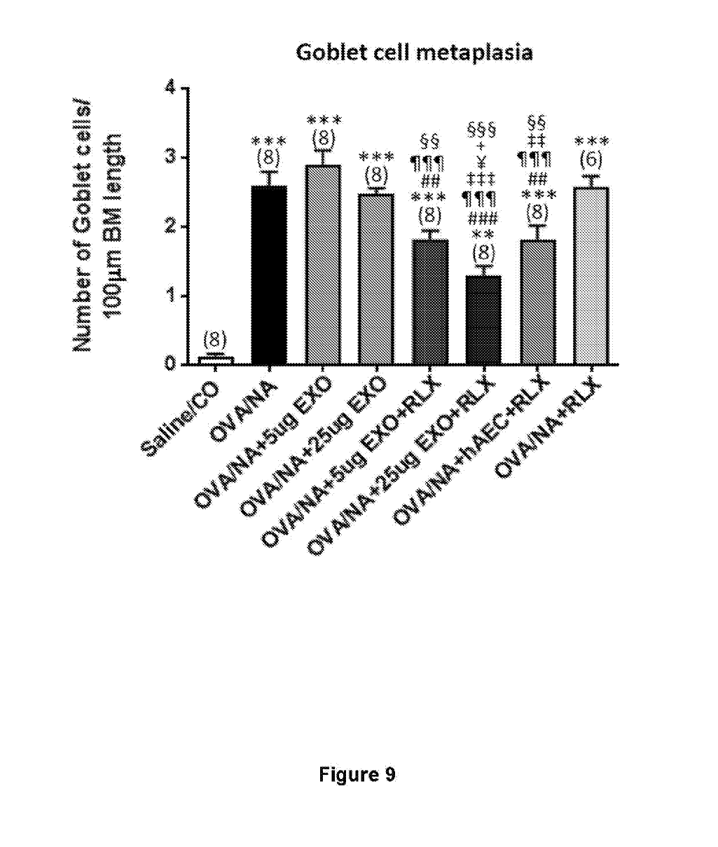

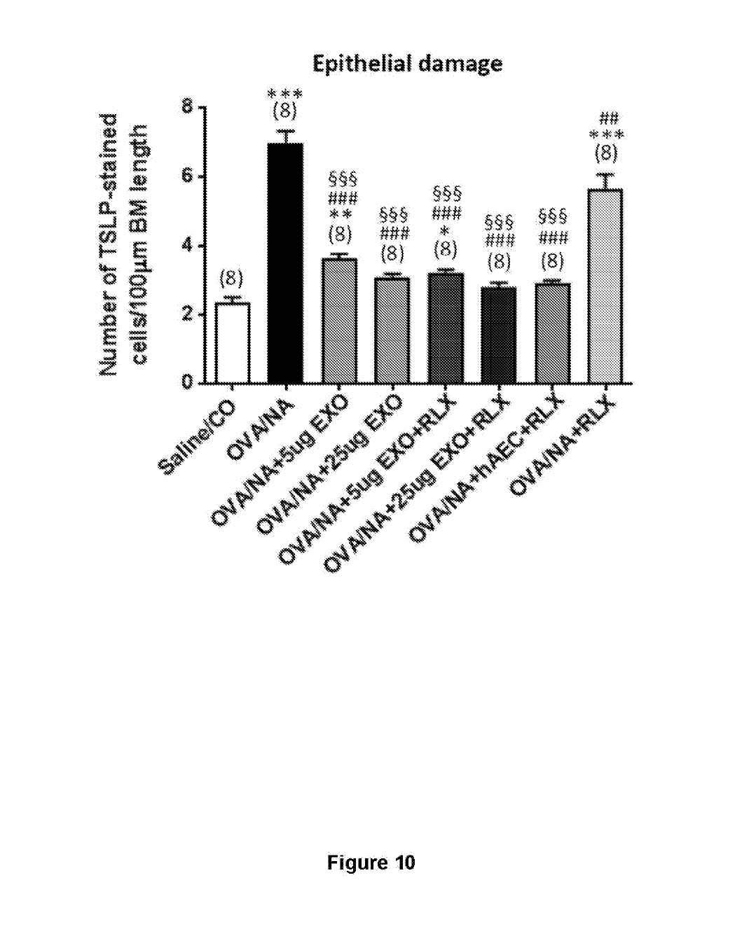

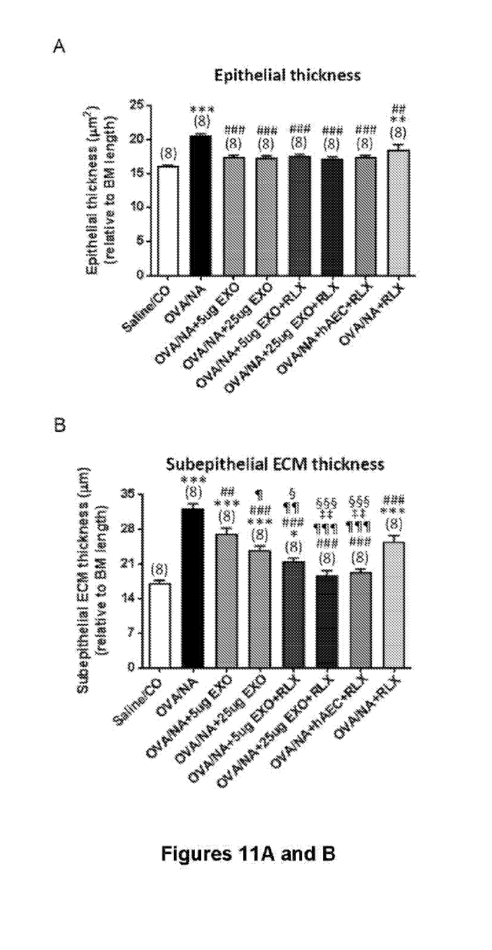

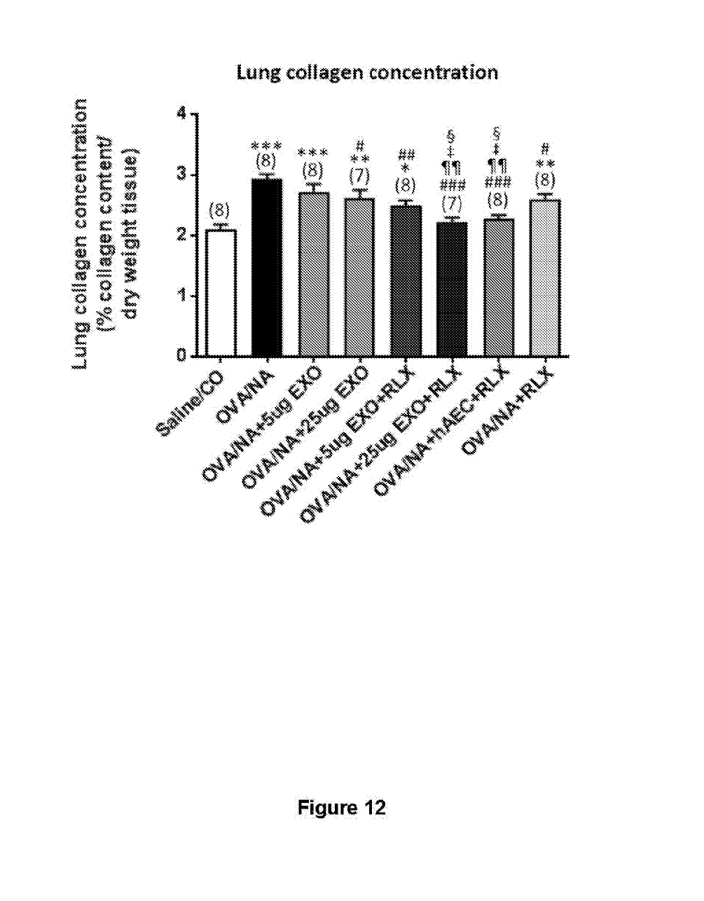

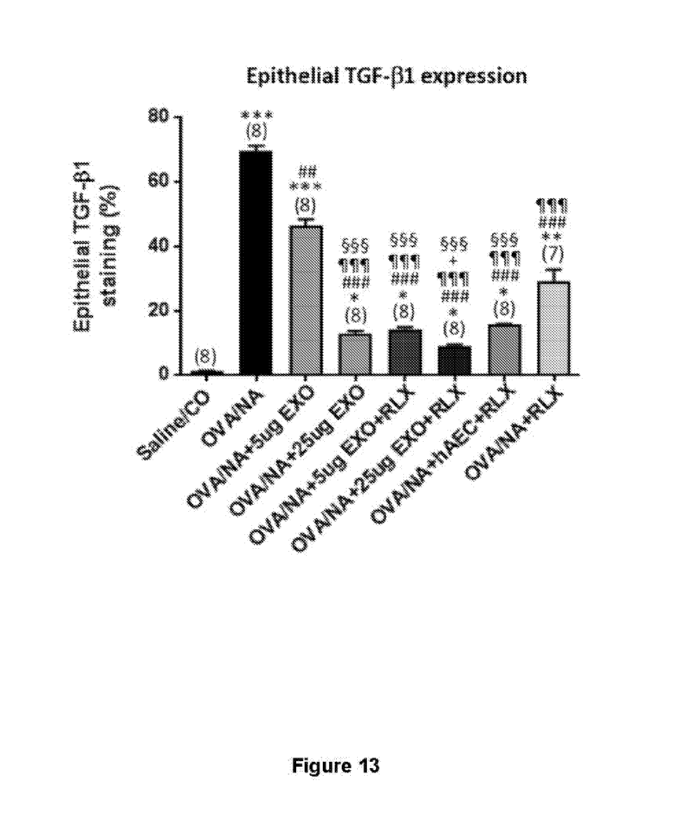

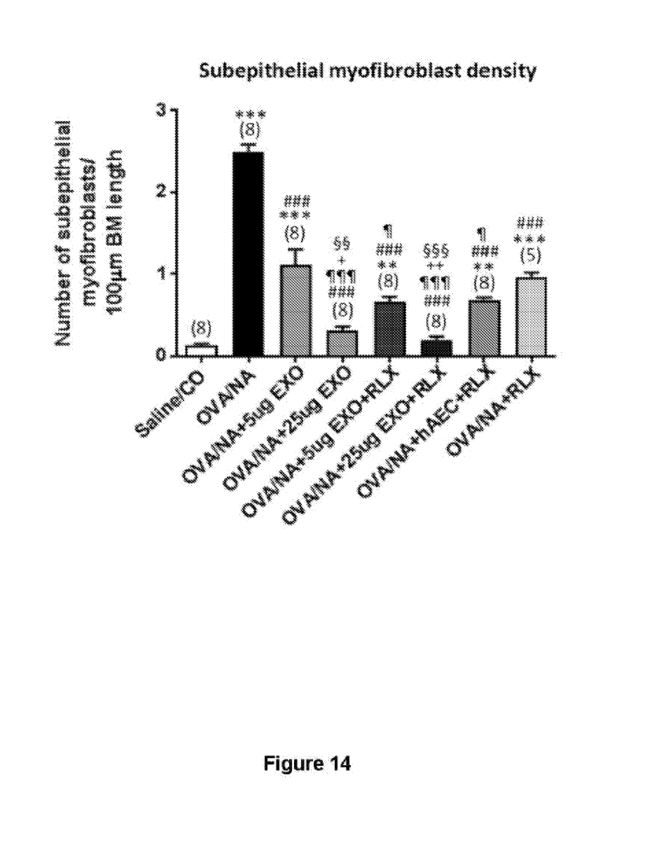

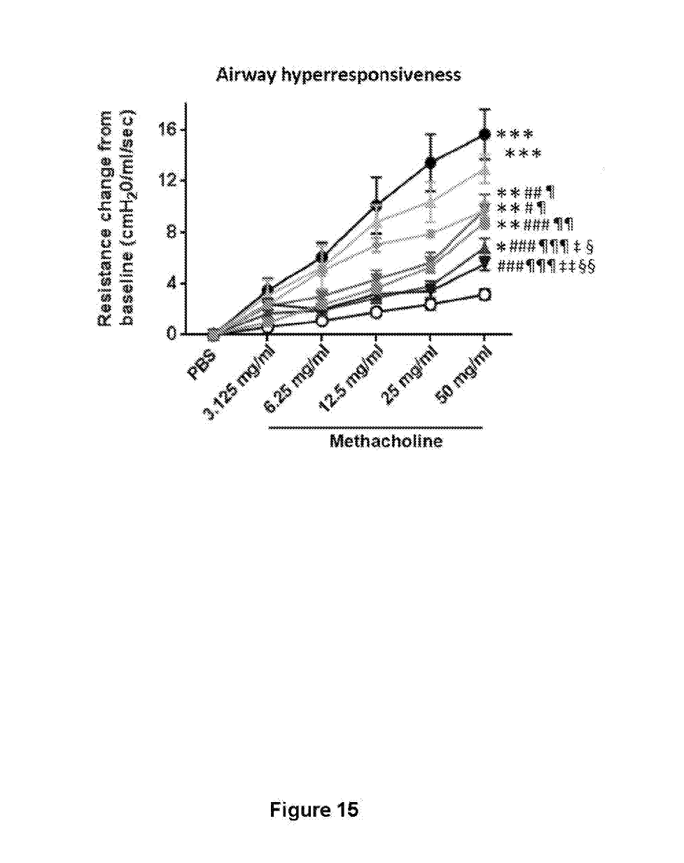

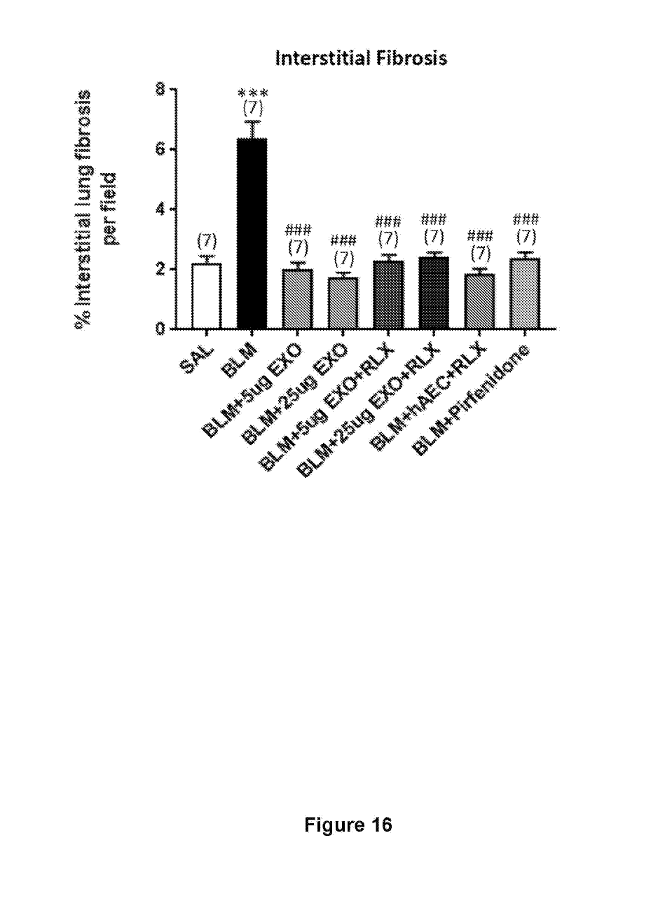

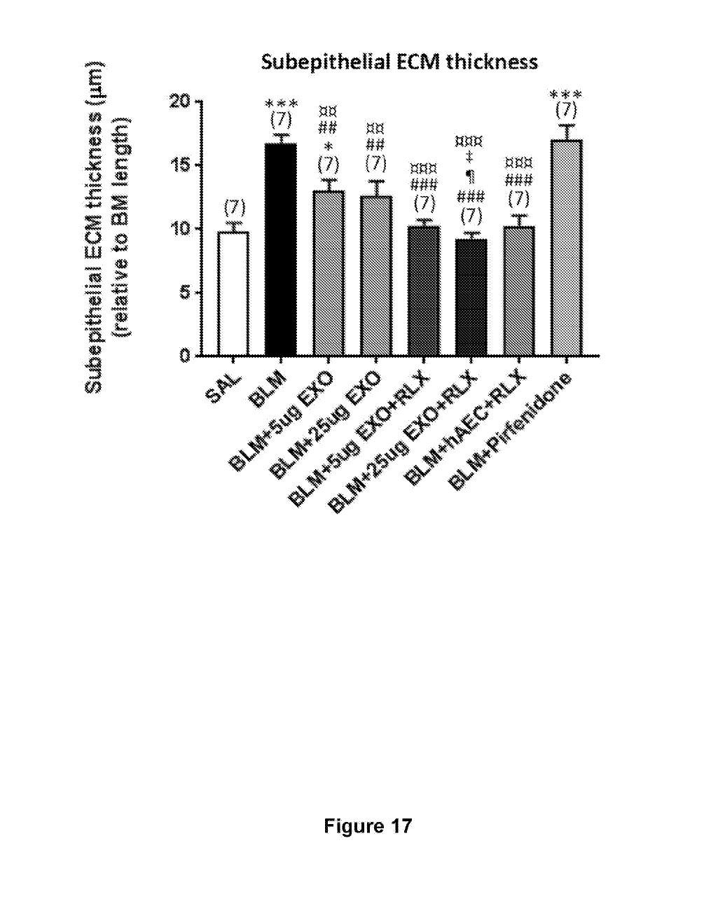

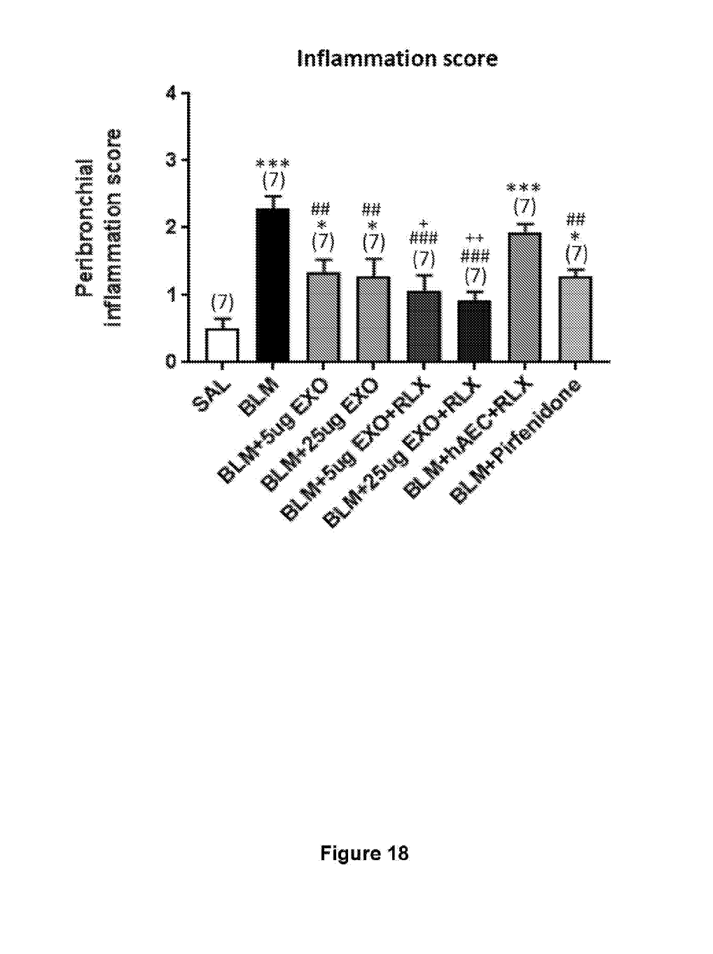

To elucidate the possible mechanisms by which the combined effects of RLX and MSCs or AECs were able to normalize the OVA-induced subepithelial and total lung collagen deposition, changes in airway epithelial TGF-β1 staining were assessed (FIGS. 6A and 6B). Epithelial TGF-β1 expression was significantly increased in OVA-treated mice (10.65±0.81), relatively compared to that in saline-treated controls (1.00±0.47; P<0.01 vs saline group; FIG. 6B). RLX alone (5.85±1.42), but not MSCs alone (8.15±1.78), was able to partially, but significantly, reduce the OVA-induced increase in aberrant epithelial TGF-β1 expression levels, as demonstrated previously in a separate study (Royce et al. (2015) supra). On the other hand, AECs alone (3.04±0.44) and the combined effects of MSCs+RLX (1.47±0.45) or AECs+RLX (2.63±0.060) were able to markedly reduce airway epithelial TGF-β1 expression to that which was no longer different to the levels measured in saline-treated controls (all P<0.001 vs OVA alone group; no difference to saline group); and to a greater extent than the effects of MSCs alone (all P<0.05 vs OVA+MSC group; FIG. 6B).

### Example 7

### Effects of RLX, MSCs, AECs and Combination Treatments on Subepithelial Myofibroblast accumulation

OVA-treatment of mice also resulted in a significantly increased number of α-SMA-stained myofibroblasts per 100 μm BM length, in the subepithelial layer of the airways (1.72±0.07) compared to that measured from their saline-treated counterparts (0.44±0.13; p<0.001 vs saline group; FIGS. 6C and 6D). All individual treatments including RLX alone (1.08±0.10), MSCs alone (1.06±0.10) or AECs alone (1.13±0.19) significantly reduced the OVA-induced increase in subepithelial myofibroblast numbers to a similar extent (by ˜45-50%; all P<0.01 vs OVA alone group; all P<0.05 vs saline group); while the combined effects of MSCs+RLX (0.88±0.12) or AECs+RLX (0.63±0.12) further reduced the number of subepithelial myofibroblasts to numbers that were no longer different to that measured in saline-treated control animals (both P<0.001 vs OVA alone group; no different to saline group; FIG. 6D).

### Example 8

### Effects of RLX, MSCs, AECs and Combination Treatments on AHR

AHR was assessed via invasive plethysmography and was significantly increased in OVA treated mice compared to that measured in saline controls (FIG. 7). AHR was partially, but significantly decreased by RLX alone (by ˜50%; P<0.001 vs OVA group; P<0.01 vs saline group) or AECs alone (by ˜35-40%; P<0.05 vs OVA group; P<0.001 vs saline group), but not MSCs alone (P<0.001 vs saline group); correlating with how effective these treatments were in reversing airway/lung fibrosis. In comparison, the combined effects of MSCs+RLX or AECs+RLX further reduced AHR to levels that were no longer significantly different to that measured from saline-treated control mice (both P<0.001 vs OVA alone group; no different to saline group; FIG. 7). The combined effects of MSCs+RLX or AECs+RLX also reduced AHR to a significantly greater extent than MSCs alone (both P<0.01 vs OVA+MSC group; FIG. 7).

### Example 9

### Chronic Allergic Airways Disease/Asthma Model Incorporating Epithelial Damage

The data presented in FIGS. 8 to 15 clearly show that the presence of relaxin augments the therapeutic potential of exosomes in an animal model of chronic allergic airways disease/asthma model which incorporates epithelial damage. This animal model is described in the section just prior to Example 1. The results show that the exosome/relaxin combinations significantly reduce inflammation score, goblet cell metaplasia, epithelial damage, epithelial thickness and sub-epithelial ECM thickness, collagen concentration, epithelial TGF-β31 expression levels, subepithelial myofibroblast density and improve airway hyperresponsiveness. Collectively, these results are consistent with those showing relaxin augments the therapeutic potential of AECs and exosomes (Examples 2 to 8).

Hematoxylin and eosin-stained lung sections from each group studies demonstrated the extent of inflammatory cell infiltration within the bronchial wall. Mean±S.E.M peribronchial inflammation score from 5 airways/mouse (where sections were scored based on the number and distribution of inflammatory cell aggregates on a scale of 0 (no visible inflammation) to 4 (severe inflammation)) is shown in FIG. 8.

Alcian blue periodic acid Schiff-stained lung sections from each group studies demonstrated the extent of goblet cell metaplasia. Mean±S.E.M goblet cell count (represented as the number of goblet cells per 100 mm basement membrane (BM) length) from 5 airways/mouse, is shown in FIG. 9.

Immunohistochemically-stained lung sections showed thymic stromal lymphopoietin (TSLP; a marker of epithelial damage) from each group studied. Mean±S.E.M TSLP-stained cell counts (per 100 μm basement membrane (BM) length) from 5 airways/mouse, is shown in FIG. 10.

Masson's trichrome-stained lung sections demonstrated the extent of epithelial thickness and subepithelial extracellular matrix (ECM/blue staining) thickness from each group studied. Mean±S.E.M (A) epithelial thickness (μm²; relative to basement membrane (BM) length); and (B) subepithelial ECM thickness (μm; relative to BM length—a measure of fibrosis) from 5 airways/mouse, is shown in FIG. 11.

Mean±S.E.M total lung collagen concentration (% lung collagen content/dry weight tissue—a measure of fibrosis); from n7-8 animals per group, is shown in FIG. 12.

Immunohistochemically-stained lung sections showed epithelial TGF-β31 expression levels from each group studied. Mean±S.E.M TGF-β1-staining (expressing as % staining per area analyzed) from 5 airways/mouse; n=7-8 animals per group. *p<0.05, **P<0.01, ***P<0.001 vs Saline/corn oil-treated uninjured control group, is shown in FIG. 13.

Immunohistochemically-stained lung sections showed subepithelial myofibroblast density. Mean±S.E.M number of α-smooth muscle actin-stained myofibroblasts in the subepithelial region (per 100 μm basement membrane (BM) length) from 5 airways/mouse, is shown in FIG. 14.

The effect of the various groups evaluated on airway hyperresponsiveness (AHR), is shown in FIG. 15. Airway resistance (reflecting changes in AHR) was assessed via invasive plethysmography in response to increasing doses of nebulized methacholine (a bronchoconstrictor). Results in FIG. 15 are expressed as resistance change from baseline. Shown is the mean±S.E.M. airway resistance to each dose of methacholine testes (n=5 animals per group). The effects of hAECs +recombinant human relaxin (RLX) or RLX alone are included for comparison.

### Example 10

### Bleomycin Interstitial Fibrosis Model

Representative images of Masson's trichrome-stained lung sections from each group studied demonstrated the extent of interstitial lung extracellular matrix/collagen deposition (fibrosis). Mean±S.E.M % interstitial fibrosis per field from 5 fields/mouse; from n=7 animals per group, is shown in FIG. 16.

Representative images of Masson's trichrome-stained lung sections from each group studied demonstrated the extent of subepithelial extracellular matrix (ECM) deposition (as another measure of fibrosis). Mean±S.E.M subepithelial ECM thickness (μm; relative to BM length) from 5 airways/mouse; n=7 animals per group, is shown in FIG. 17.

The fibrosis data from the bleomycin model showed that while all treatments (including pirfenidone) normalized the bleomycin-induced increased in interstitial fibrosis (FIG. 16), that only RLX+exosomes (EXO) or RLX+hAECs were able to also normalize the bleomycin-induced increase in subepithelial ECM/collagen deposition as well (FIG. 17). In comparison, 5 μm exosomes alone only had a partial effect in reversing subepithelial collagen deposition while pirfenidone had no effect. The trends are similar in showing that 25 μg EXO+RLX provides optimal protection and reverses both interstitial and subepithelial collagen deposition back to that measured in saline-treated control mice. The data support the treatment of pulmonary fibrosis such as idiopathic pulmonary fibrosis.

### Example 11

### Reversal of Bleomycin-Induced Lung Inflammation

Inflammation score from the bleomycin model (FIG. 18) shows that exosomes alone or pirfenidone alone only partially reduce bleomycin-induced lung inflammation, whereas the combined effects of exosomes +relaxin are able to fully reverse bleomycin-induced lung inflammation. Hematoxylin and eosin stained lung sections demonstrated the extent of inflammatory cell infiltration with bronchial wall.

The bleomycin model (BLM model) presents with interstitial and to a lesser extent peribronchial inflammation whereas the AAD model primarily presents with peribronchial inflammation. The data support the conclusion that AECs+relaxin were unable to limit BLM-induced lung inflammation; which may be explained by the fact that AECs have a limited time to produce exosomes and elicit an effect before being cleared from the body. The data also show that the exosomes can directly exert their effect on target cells by bypassing the cell priming step and subsequent lag before exosome biogenesis occurs. BLM-induced lung inflammation was optimally reduced by the combined effects of exosomes +relaxin, to levels that were not significantly different to that measured in the saline-treated uninjured control group.

### Example 12

### Development of Therapeutic Protocol

The previous Examples compare the therapeutic effects of AECs to that of MSCs, in the absence and presence of an anti-fibrotic agent (RLX), in the setting of chronic OVA-induced AAD. The results show that: 1) AECs alone demonstrated greater protection against the chronic AAD-induced increase in AI (as assessed by changes in peribronchial inflammation score and eosinophil counts), AWR (as assessed by changes in epithelial damage/thickness, total lung collagen concentration and epithelial TGF-β1 expression) and AHR, compared to the effects of the MSCs alone. However, AEC-treatment alone was not able to fully reverse the structural and functional changes associated with the model. 2) The presence of RLX was able to enhance the protection offered by MSCs or AECs, such that eosinophil counts, epithelial thickness, collagen deposition, epithelial TGFIβ1 expression levels, subepithelial myofibroblast accumulation and AHR were all normalized in both combination treatment groups, returning to that measured in saline-treated control mice. The superior anti-fibrotic effects of the combination treatments appeared to be explained by their greater ability to reverse aberrant TGF-β1 expression and myofibroblast differentiation, compared to the effects of the individual treatments. Hence, in accordance with the present invention, combining RLX with AECs effectively reverses several aspects of AWR (including airway/lung fibrosis), and the AWR-induced changes in AHR in the setting of chronic AAD.

Although all treatments evaluated were able to markedly reduce or normalize eosinophil infiltration, RLX and MSCs alone were less effective in reversing peribronchial inflammation in general. In comparison, AECs alone demonstrated the most effective anti-inflammatory effects and also markedly reduced eosinophil counts to a level which was no longer different to that measured in saline-treated controls. Interestingly, the combined effects of AECs and RLX further reduced peribronchial inflammation score to a greater extent than RLX or MSCs alone. In the chronic AAD model, intratracheal transplantation of MSCs was shown to decrease infiltration of eosinophils, neutrophils and monocytes within the BAL fluid (Ge et al. (2013) supra). The effects of AECs have not been investigated before in the setting of chronic AAD. RLX alone has not been effective in reducing the infiltration of monocytes (Royce et al. 92009) supra; Royce et al. (2014) supra) and lymphocytes (Royce et al. (2009) supra; Royce et al. (2014) supra) in the chronic mouse model of AAD, and macrophages in other models (Samuel et al. (2011) Lab. Invest. 91:675-690). The combined effects of RLX with AECs, have broader anti-inflammatory effects compared to other treatments.

The infiltration of these various inflammatory cells, as well as Th2 CD4⁺ cells, is also associated with various structural changes that encompass AWR. During asthma, repeated cycles of injury and repair lead to aberrant structural changes in the airways which contribute to an irreversible loss of lung function (Hirota and Martin (2013) Chest 144:1026-1032). In the current Examples, AWR was assessed via epithelial thickening, goblet cell metaplasia, airway collagen deposition (fibrosis) as well as epithelial TGF-β1 expression and subepithelial myofibroblast accumulation.

Epithelial thickening contributes to narrowing of the airways and, hence, increased airway resistance, resulting in an increase in asthma-induced breathing difficulties (Hogg (1997) APMIS 105:745-745). Epithelial thickening was exacerbated in the OVA-treated mice and was not significantly affected by MSC alone-treatment. This is consistent with previous results demonstrating an inability of intranasally- (Royce et al. (2015) supra) or intravenously (i.v.) [Firinci et al. (2011) Int. Immunopharmacol. 11:1120-1126]-delivered MSCs to inhibit epithelial thickening in the chronic AAD model. In contrast, RLX alone was able to reduce epithelial thickness, which is consistent with previous findings using chronic AAD-injured mice treated with RLX systemically (Royce et al (2009) supra) or intranasally (Royce et al. (2014) supra). Likewise, AECs alone and in combination with RLX were found to normalize the OVA-induced increased in epithelial thickness. These findings may suggest that treatments which can normalize aberrant epithelial thickening and the related fibrosis that ensues from epithelial damage to the airways/lung are more likely to protect from AAD induced AWR and the contributions of AWR to AHR. Indeed another recent study demonstrated that the ability of RLX alone or in combination with an epithelial repair factor (trefoil factor-2; TFF2) or with TFF2 and the corticosteroid, dexamethasone, was able to completely reverse the OVA-induced loss of dynamic lung compliance, due to the ability of these treatments to significantly reduce both epithelial thickness and lung collagen concentration (the basis of fibrosis) [Patel et al. (2016) Br. J. Pharmacol. doi:10.1111/bph.13494].

Related to epithelial thickening, the present invention evaluates a marker of lung damage (TSLP), which is markedly produced and secreted by airway epithelial cells (along with IL-25 and IL-33) in response to various stimuli of asthma-like symptoms (Bartemes and Kita (2012) Clin. Immunol. 143:222-235). Interestingly, all treatments that could significantly reverse epithelial thickening were found to also partially reduce the OVA-induced increase in airway epithelial TSLP expression. These findings imply that administration of RLX or AECs alone and the combination treatments evaluated were able to partially induce epithelial tissue repair. Consistent with the findings of this study, RLX alone and in combination with MSCs were also found to significantly suppress epithelial kidney injury molecule (KIM-1) expression in a ureteric obstruction-induced model of kidney disease/fibrosis (Huuskes et al. (2015) supra). This may have been due to the anti-apoptotic and/or angiogenic effects of these therapies (Samuel et al. (2011) supra; Linthout et al. (2011) Curr. Pharmaceut. Des. 17:3341-3347).

Deposition of excessive ECM components, particularly collagens, occurs within the subepithelial and adventitia of airways in asthmatic individuals and contributes to the development of fibrosis (Gillis and Lutchen (1999) J. Appl. Physiol. 86:2011-2012). In the current Examples, fibrosis was evaluated by examining subepithelial and total collagen content, as well as two markers of collagen turnover, namely TGF-β1 and α-SMA (a marker of myofibroblast differentiation). MSCs alone were able to partially reduce subepithelial collagen levels but only demonstrated a trend towards reducing total lung collagen concentration levels, which is somewhat inconsistent with previous studies (Royce et al. (2014) supra; Royce et al. (2014) supra) and suggestive that the effects of these cells may be patient- and/or batch-to-batch dependent. In comparison, either RLX or AECs alone were able to significantly although not totally reverse the OVA-induced increase in both subepithelial and total lung collagen. This is consistent with previous studies which have shown that RLX alone can reduce airway/lung fibrosis associated with chronic AAD (Royce et al. (2009) supra; Royce et al. (2014) supra; Royce et al. (2015) supra); and those showing that AECs alone (Moodley et al. (2010) supra; Murphy et al. (2011) supra) can reduce interstitial lung fibrosis following bleomycin-induced injury. More impressively, both combination treatments were able to further reduce and in fact normalise the OVA induced increase in airway/lung fibrosis. This is also consistent with previous findings using the combination of MSCs and RLX to reduce both the renal fibrosis induced by unilateral ureteral obstruction (Huuskes et al. (2015) supra).

In the current Examples, the limited anti-fibrotic efficacy demonstrated by MSCs in the setting of chronic AAD was found to be associated with their lack of ability to affect airway epithelial TGF-β1 expression levels, while modestly reducing subepithelial myofibroblast accumulation. The anti-fibrotic efficacy demonstrated by RLX or AECs alone was consistent with their ability to significantly reduce both airway epithelial TGF-β1 expression levels and subepithelial myofibroblast accumulation; and with the general TGF-β1-inhibitory effects of both RLX (Unemori et al. (1996) supra; Tozzi et al. (2005) supra; Patel et al. (2016) supra) and AECs (Moodley et al. (2010) supra; Moodley et al. (2013) PLoS ONE8;e69299) in the airways/lung. Of further significance, the enhanced anti-fibrotic efficacy of both combination therapies resulted in their ability to both normalise the aberrant increase in chronic AAD-induced epithelial TGF-β1 expression levels, while markedly lowering subepithelial myofibroblast differentiation; likely leading to the marked reversal, if not normalization of the chronic AAD-induced increase in aberrant subepithelial and total lung collagen deposition measured. These findings indicate that RLX can be combined with multiple stem cells types that express RXFP1, to treat the AWR and related AHR associated with asthma; either by creating a more favourable environment in which these transplanted cells can survive and execute their therapeutic/reparative effects and/or by directly promoting their viability, proliferation and migration.

Both AI and AWR contribute to the development of AHR (Ober and Hoffjan (2006) Genes Immun. 7:95-100; Kariyawasam et al. (2007) Am. J. Respir. Crit. Care Med. 175:896-904). The extent of AHR also relates to the severity of asthma (Bourlet et al. (1997) Chest 112:45-52, Holgate et al. (2004) Proc. Am. Thorac. Soc. 1:93-98). Previous studies have demonstrated that an increase in BM thickness due to deposition of ECM components (subepithelial fibrosis) correlates with a decrease in distensibility of the airways (Ward et al. (2001) Am. J. Respir. Crit. Care Med. 164:1718-1721), concurrent with an increase in AHR (Milanese et al. (2001) J. Appl. Physiol. 91:1035-1040). In the current study, AHR was evaluated using invasive plethysmography to determine airway resistance. The OVA-induced increase in AHR was proportionally reduced in line with how effective each of the treatments investigated reversed airway/lung fibrosis; with either of the combination treatments being able to completely reverse AHR back to levels measured from saline-treated control mice, based on their ability to normalise airway epithelial thickness, epithelial TGF-β1 expression levels and total lung collagen concentration. These findings are consistent with published study (Patel et al. (2016) supra) which also demonstrated that other therapies that could abrogate the chronic AAD-induced aberrant increase in epithelial thickness and/or damage as well as collagen concentration, was key to normalising methacholine induced AHR. However, the extent to which these treatments reduced airway/lung fibrosis appeared to correlate more with how effectively they could reverse AHR.

The present invention is predicated in part on the findings that combining RLX with AECs or exosomes was able to significantly reduce AI, and completely abrogate airway epithelial thickening, airway epithelial TGF-β1 expression levels, airway/lung fibrosis and AHR associated with chronic AAD. Hence, this combination therapy offers a novel means to treat the three central components of airways disease pathogenesis (AI, AWR and AHR), particularly to patients who are resistant to corticosteroid therapy. AECs in combination with RLX were effective in further reducing AI and AWR as well as improving AHR associated with asthma.

Accordingly, the therapeutic protocol comprises the administration to a subject in need of therapy:
(A) (i) AECs; or
(ii) amniotic exosomes; and
(B) an anti-fibrotic agent such as relaxin or a recombinant or derivative form.

In an embodiment, the therapeutic protocol comprises the administration to a subject in need of therapy of amniotic exosomes and an anti-fibrotic agent such as relaxin or a recombinant or derivative form.

The administration is by any convenient route such as intranasal, respiratory, intratracheal, nasopharyngeal, intravenous, intraperitoneal, intrathoracic, subcutaneous, intradermal, intramuscular, intraocular, intrathecal or rectal routes. The addition of an activator or agonist of a receptor via which the anti-fibrotic agent acts (e.g. RXFP1 for relaxin) may also assist. The therapy is proposed to ameliorate any or all of AI, AWR and/or AHR and reduce fibrosis. The AECs or exosomes and the anti-fibrotic agent may be co-administered or separately or sequentially in either order or co-formulated together.

As taught in the present Examples, the combination of AECs or exosomes and relaxin is found to be more efficacious than a combination of mesenchymal stem cells (MSCs) and relaxin. AECs or exosomes and relaxin normalize epithelial thickness and partially reverse fibrosis in a mouse model as well as ameliorate airway hyper-responsiveness. It is proposed herein that the anti-fibrotic agent such as relaxin provides an improved environment in which AEC-based therapies can be employed, enhancing the therapeutic and regenerative capacity of AECs expressing the relaxin-2 receptor, RXFP1. It is further proposed that the therapeutic benefits of AECs apply to amniotic exosomes. In this regard, the amniotic exosomes may be isolated from amniotic fluid or placental tissue or may be isolated from AEC lines including immortalized AEC lines. This includes the use of a bioreactor to generate amnion exosomes from AEC lines which are subsequently isolated and used in the present therapeutic protocol. Notwithstanding, the present invention extends to the use of MSCs and an anti-fibrotic agent such as relaxin to treat airways disease.

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

### BIBLIOGRAPHY

Agoulnik et al. (2017) British Journal of Pharmacology 174:977-989
Akle et al. (1981) Lancet 2:1003-1005
Baba et al. (1993) J. Neurosurg. 79:729-735
Baraniak and McDevitt (2010) Regen. Med. 5:121-143
Bartemes and Kita (2012) Clin. Immunol. 143:222-235
Berkner (1988) Biotechniques 6:616-627
Bourlet et al. (1997) Chest 112:45-52
Braman (2006) Chest 130:4S-12S
Connelly (1995) Human Gene Therapy 1:185-193
Curiel (1992) Hum. Gene Ther. 3:147-154
Dolgachev et al. (2009) Am J. Path. 174:390-400
Firinci et al. (2011) Int. Immunopharmacol. 11:1120-1126
Flotte et al. (1993) Proc. Natl. Acad. Sci. USA 90:10613-10617
Gallop and Paz (1975) Physiol. Revs. 55:418-487
Ge et al. (2013) J. Cell. Biochem. 114:1595-1605
Gillis and Lutchen (1999) J. Appl. Physiol. 86:2011-2012
Ha et al. (2016) Acta PharmaceuticaSinicaB 6(4):287-296
Harlow, Lane and Harlow, Using Antibodies: A Laboratory Manual: Portable Protocol NO. I, Cold Spring Harbor Laboratory (1998)
Harlow and Lane, Antibodies: A Laboratory Manual, Cold Spring Harbor Laboratory; (1988)
Hirota and Martin (2013) Chest 144:1026-1032
Hogg (1997) APMIS 105:745-745
Holgate et al. (2004) Proc. Am. Thorac. Soc. 1:93-98
Holgate (2008) Clin. Exp. Allergy 38:872-897
Hossain et al. (2011) J. Biol. Chem. 286 :37555-37565
Hossain et al. (2016) Chem. Sci. 7:3805-3819
Hsu et al. (2002) Science 295:671-674
Huang et al. (2011) Am. J. Path. 179:2751-2765
Huuskes et al. (2015) FASEBI J. 29:540-553
Ivell (2002) Science 295:637-638
Jolly (1994) Cancer Gene Therapy 1:51-64
Kaplitt (1994) Nature Genetics 6:148-153
Kariyawasam et al. (2007) Am. J. Respir. Crit. Care Med. 175:896-904
Kass-Eisler et al. (1993) Proc. Natl. Acad. Sci. USA 90:11498-11502
Kenyon et al. (2003) Toxicol. Appl. Pharmacol. 186:90-100
Kimura (1994) Human Gene Therapy 5:845-852
Knight and Rossi (2010) Expert Rev. Respir. Med. 4:747-758
Kolls et al. (1994) Proc. Natl. Acad. Sci. USA 91:215-219
Levey et al. (2006) Prim. Care Respir. J. 15:20-34
Linthout et al. (2011) Curr. Pharmaceut. Des. 17:3341-3347
Marceau et al. (2006) J. Allergy Clin. Immunol. 118:574-581
Maniatis, Fritsch & Sambrook, Molecular Cloning: A Laboratory Manual (1982)
Mendelson et al. (1988) Virol. 166:154-165
Miki et al. (2005) Stem Cells 23:1549-1559
Milanese et al. (2001) J. Appl. Physiol. 91:1035-1040
Moodley et al. (2010) Am. J. Rspir. Crit. Cre Med. 182:643-651
Murphy et al. (2010) Curr. Prot. Stem Cell Biol. chapter 1, Unit 1E6]
Myers and Tomasio (2011) Respir. Care 56:1389-1407
Ober and Hoffjan (2006) Genes Immun. 7:95-100
Patel et al. (2016) Br. J. Pharmacol. doi: 10.1111/bph.13494
Patil et al. (2017) British Journal of Pharmacology 174(10):950-961
Philip (1994) Mol. Cell Biol. 14:2411-2418
Putney and Burke (1998) Nat Biotech 16:153-157
Ram et al. (1993) Cancer Res. 53:83-88
Remington: The Science and Practice of Pharmacy 21^{st} ed. (2006)
Rosenfeld et al. (1991) Science 252:431-434
Royce and Tang (2009) Curr. Mol. Pharmacol 2:169-181
Royce et al. (2009) Endocrinology 150:2692-2699
Royce et al. (2014) Pharm. therap. 141:250-260
Royce et al. (2015) Stem Cell Res. 15:495-505
Royce et al. (2016) Clinical Science 130:3151-65
Sambrook, Russell and Sambrook, Molecular Cloning: A Laboratory Manual (2001)
Samuel et al. (2007) Cell. Mol. Life Sci. 64:1539-1557
Samuel et al. (2011) Lab. Invest. 91:675-690
Samuel et al. (2017) British Journal of Pharmacology 174:962-976
Samulski et al. (1989) J. Vir. 63:3822-3828
Stanojevic et al. (2012) BMC Public Health 12:204
Sterzenbach et al. (2017) Molecular Therapy 25(6):1269-1278
Sun et al. (2012) Stem Cells 30:2692-2699
Takamiya et al. (1992) J. Neurosci. Res. 33:493-503
Teerlink et al. (2013) Lancet 281:29-39
Tozzi et al. (2005) Pulm. Pharm. Therap. 18:346-353
Unemori et al. (1996) J. Clin. Invest. 98:2739-2745
Vile and Hart (1993) Cancer Res. 53:3860-3864
Vile and Hart (1993) Cancer Res. 53:962-967
Ward et al. (2001) Am. J. Respir. Crit. Care Med. 164:1718-1721
Weiss (2014) Stem Cells 32:16-25
Wise et al. (2014) am. J. Phyisol. Renal Physiol. 306:F1222-F1235
Woffendin (1994) Proc. Natl. Acad. Sci. 91:1581-1585

1. A method for treating airways disease in a subject, said method comprising administering to said subject, a therapeutically effective amount of amnion epithelial cells (AECs) or amniotic exosomes together with an anti-fibrotic agent, the treatment being for a time and under conditions sufficient to ameliorate one or more of airway inflammation, airway remodeling and/or airway hyper-responsiveness.

2. The method of claim 1 wherein the anti-fibrotic agent is relaxin-2, a recombinant form of relaxin-2 or a functional derivative or variant or mimetic of relaxin-2.

3. The method of claim 2 wherein the recombinant form of relaxin-2 is serelaxin.

4. The method of claim 3 wherein the derivative of relaxin-2 is a single B chain derivative or an A and B chain truncate of relaxin-2.

5. The method of claim 4 wherein the single B chain derivative of relaxin-2 is H2-(B7-33) or the A and B chain truncate is H2-(A4-24)(B7-24).

6. The method of claim 2 further comprising the administration of an RXFP1 activating agent or agonist.

7. The method of claim 2 wherein the AECs are genetically modified to express the relaxin or its derivative or variant.

8. The method of claim 2 wherein the amniotic exosomes are modified to contain the relaxin or its recombinant or derivative or variant form.

9. The method of claim 1 wherein the subject is a human.

10. The method of claim 1 wherein the subject is a racing animal.

11. The method of claim 10 wherein the racing animal is a horse.

12. The method of claim 2 wherein the AECs or exosomes are co-administered simultaneously or sequentially with the relaxin or the AECs or exosomes comprise the relaxin.

13. The method of claim 12 wherein the relaxin is administered followed by the AECs or amniotic exosomes.

14. The method of claim 1 wherein administration of one or other or both of the relaxin and the AECs or exosomes is by intranasal, intrarespiratory, intranasopharyngeal or intravenous administration.

15. The method of claim 1 wherein the AECs are autologous or allogenic or xenogeneic to be subject being treated.

16. The method of claim 1 wherein the exosomes are autologous or allogeneic or xenogeneic to the subject being treated.

17. The method of claim 2 wherein the relaxin is autologous or allogeneic or xenogeneic to the subject being treated.

18. The method of claim 1 wherein the airways disease is selected from the list consisting of asthma, allergic rhinitis, chronic obstructive pulmonary disease, pulmonary fibrosis, upper respiratory infection and reactive airways dysfunction syndrome.

19. The method of claim 18 wherein the pulmonary fibrosis is idiopathic pulmonary fibrosis or other interstitial lung disease.

20. The method of claim 18 wherein the airways disease is asthma.

21. The method of claim 1 in the treatment, prevention or reduction in lung tissue or respiratory tissue fibrosis.

22. A pharmaceutical kit comprising in compartmental form a first compartment comprising AECs or amniotic exosomes in a form which can be reconstituted in a pharmaceutically acceptable medium; a second compartment comprising an anti-fibrotic agent for use with lung tissue; wherein the AECs or exosomes are reconstituted in the pharmaceutically acceptable medium prior to use wherein the AECs and anti-fibrotic agent are administered to a subject simultaneously or sequentially in either order.

23. A formulation comprising AECs or amniotic exosomes and an anti-fibrotic agent and one or more pharmaceutically acceptable carriers, excipients and/or diluents.

24. The pharmaceutical kit of claim 22 or formulation of claim 23 wherein the anti-fibrotic agent is relaxin-2, a recombinant form of relaxin-2 or a functional derivative of relaxin-2.

25. The pharmaceutical kit or formulation of claim 24 wherein the recombinant form of relaxin-2 is serelaxin.

26. The pharmaceutical kit or formulation of claim 24 wherein the derivative of relaxin-2 is a single B chain derivative or an A and B chain truncate of relaxin-2.

27. The pharmaceutical kit or formulation of claim 26 wherein the single B chain derivative of relaxin-2 is H2-(B7-33) or the A and B chain truncate is H2-(A4-24)(67-24).

28. The pharmaceutical kit of claim 22 or formulation of claim 23 further comprising an RXFP1 activating agent or agonist.

29. The pharmaceutical kit of claim 22 or a formulation of claim 23 alternatively comprising AECs or exosomes containing the anti-fibrotic agent.

30. The pharmaceutical kit or formulation of claim 29 wherein the anti-fibrotic agent is relaxin or a recombinant or a functional derivative thereof.

31. A use of AECs or amniotic exosomes in combination with an anti-fibrotic agent in the manufacture of a medicament for the treatment of airways disease in a subject.

32. The use of claim 31 wherein the anti-fibrotic agent is relaxin-2, a recombinant form of relaxin-2 or a functional derivative of relaxin-2.

33. The use of claim 31 wherein the airways disease is selected from the list consisting of asthma, allergic rhinitis, chronic obstructive pulmonary disease, pulmonary fibrosis, upper respiratory infection and reactive airways dysfunction syndrome.

34. The use of claim 33 wherein the pulmonary fibrosis is idiopathic pulmonary fibrosis or other interstitial lung disease.

35. The use of claim 33 wherein the airways disease is asthma.

36. The use of claim 31 in the treatment, prevention or reduction in lung tissue or respiratory tissue fibrosis.

37. AECs or amniotic exosomes and an anti-fibrotic agent for use in the treatment of airways disease in a subject.

38. The AECs or amniotic exosomes and an anti-fibrotic agent of claim 37 wherein the anti-fibrotic agent is relaxin-2, a recombinant form of relaxin-2 or a functional derivative of relaxin-2.

39. The AECs or amniotic exosomes and anti-fibrotic agent of claim 37 wherein the airways disease is selected from the list consisting of asthma, allergic rhinitis, chronic obstructive pulmonary disease (COPD), pulmonary fibrosis, upper respiratory infection and reactive airways dysfunction syndrome.

40. The AECs or amniotic exosomes and anti-fibrotic agent of claim 39 wherein the pulmonary fibrosis is idiopathic pulmonary fibrosis or other interstitial lung disease.

41. The AECs or amniotic exosome and anti-fibrotic agent of claim 39 wherein the airways disease is asthma.

42. The AECs or amniotic exosome and anti-fibrotic agent of claim 37 in the treatment, prevention or reduction in lung tissue or respiratory tissue fibrosis.

43. A method of treating airways disease in a subject, the method comprising administering to the subject, a therapeutically effective amount of mesenchymal stem cells (MSCs) together with an anti-fibrotic agent, the treatment being for a time and under conditions to ameliorate one or more of airway inflammation, airway remodeling and/or airway hyperresponsiveness.

44. The method of claim 43 wherein the anti-fibrotic agent is relaxin-2, a recombinant form of relaxin-2 or a functional derivative or variant or mimetic of relaxin-2.

45. The method of claim 44 wherein the recombinant form of relaxin-2 is serelaxin.

46. The method of claim 45 wherein the derivative of relaxin-2 is a single B chain derivative or an A and B chain truncate of relaxin-2.

47. The method of claim 46 wherein the single B chain derivative of relaxin-2 is H2-(B7-33) or the A and B chain truncate is H2-(A4-24)(B7-24).

48. The method of claim 43 wherein the subject is a human.

49. The method of claim 43 wherein the subject is a racing animal.

50. The method of claim 43 wherein the airways disease is selected from the list consisting of asthma, allergic rhinitis, chronic obstructive pulmonary disease, pulmonary fibrosis, upper respiratory infection and reactive airways dysfunction syndrome.

51. The method of claim 50 wherein the pulmonary fibrosis is idiopathic pulmonary fibrosis or other interstitial lung disease.

52. A formulation comprising mesenchymal stem cells (MSCs) and an anti-fibrotic agent and one or more pharmaceutically acceptable carriers, excipients and/or diluents.

53. The formulation of claim 52 wherein the anti-fibrotic agent is a relaxin.
